(19) United States
(12) Patent Application Publication
(10) Pub. No.: US 2019/0329209 A1
(43) Pub. Date: Oct. 31, 2019

(54) METHODS AND COMPOSITIONS FOR EMULSIFICATION OF SOLID SUPPORTS IN DEFORMABLE BEADS

(71) Applicant: Mission Bio, Inc., San Francisco, CA (US)

(72) Inventors: Adam R. Sciambi, San Francisco, CA (US); Dennis Jay Eastburn, San Mateo, CA (US); Sebastian Treusch, San Francisco, CA (US)

(73) Assignee: Mission Bio, Inc., San Francisco, CA (US)

(21) Appl. No.: 16/310,336

(22) PCT Filed: Jun. 13, 2017
(86) PCT No.: PCT/US2017/037175

§ 371 Date: Dec. 14, 2018

Related U.S. Application Data
(60) Provisional application No. 62/350,136, filed on Jun. 14, 2016.
(60) Provisional application No. 62/372,582, filed on Aug. 9, 2016.

(51) Int. Cl. B01J 13/04; B01F 3/08; B01F 13/00; C12Q 1/6806; C07K 1/04

(52) U.S. Cl. CPC B01J 13/04; B01F 3/0811; B01F 13/0062; B01F 2215/0037; C07K 1/042; B01F 2003/0846; C12Q 1/6806

(57) ABSTRACT

Disclosed herein are methods and compositions for the emulsification of solid supports in deformable gel beads. The methods and compositions provided herein may be used in microfluidic systems and devices. In some aspects of the disclosure, deformable gel beads containing solid supports may be paired with single cell entities. The methods and compositions provided herein may be suitable for single cell analysis, including, but not limited to, labeling single cells or components thereof for downstream analysis.

### CROSS-REFERENCE

This application claims the benefit of U.S. Provisional Application No. 62/350,136, filed Jun. 14, 2016, which is hereby incorporated by reference in its entirety, and this application claims benefit of U.S. Provisional Application No. 62/372,582, filed Aug. 9, 2016, which is hereby incorporated by reference in its entirety.

### BACKGROUND

Solid support beads, such as those surface-coated with oligonucleotides for use in single-cell barcoding, can be difficult to manipulate microfluidically due to their rigidity and tendency to jam in narrow channels at high concentrations. This jamming makes pairing the beads with other components found in microfluidics (e.g. droplets or cells) unreliable and often leads to considerable loss of resources resulting from over- or under-labeling the other components.

Droplet labeling using solid support beads, if allowed to proceed without regard to the disclosure herein, occurs such that droplet labeling events display a Poisson distribution. That is, the following statements regarding the labeling are true. The number of time that an event occurs in an interval takes a whole number. The labeling of one droplet does not affect the probability of a second labeling event. The rate of droplet labeling events is constant. Two labeling events cannot occur at exactly the same time. The probability of a labeling event in an interval is proportional to the length of the interval. Accordingly, the probability of k events occurring in an interval is described by the equation

$$P ( k events in interval ) = \frac{{λ}^{k} {e}^{- λ}}{k !} ,$$

wherein lambda is the average number of events per interval, e is Euler's number (2.718 . . . ), k takes a counting number value, and k! is k factorial, or k x(k−1) x(k−2) . . . x2 x1.

A drawback of a labeled droplet population demonstrating a Poisson distribution of labeling events is that unlabeled and multiply labeled events represent a substantial proportion of the droplets in the population. For many applications, unlabeled and multiply labeled droplets are undesirable outcomes from a labeling process because droplet contents are not easily grouped in downstream applications.

### SUMMARY

According to one aspect, a method of droplet generation, can comprise: transporting a first fluid comprising a plurality of gel beads at a controlled distance relative to one another through a first microfluidic channel, a gel bead of the plurality of gel beads comprising a solid support and a gel outer layer encapsulating the solid support; and generating a plurality of droplets comprising a number of droplets encapsulating a single gel bead at a proportion greater than 20% of the plurality of droplets, the generating comprising intersecting the first fluid with an immiscible carrier fluid.

In some embodiments, the plurality of gel beads is closely packed. In some embodiments, the number of droplets is greater than 30% of the plurality of droplets. In some embodiments, the number of droplets is greater than 40% of the plurality of droplets. In some embodiments, the number of droplets is greater than 50% of the plurality of droplets. In some embodiments, a gel bead of the plurality of closely packed gel beads is in contact with at least one other gel bead of the plurality of closely packed gel beads. In some embodiments, a gel bead of the plurality of closely packed gel beads is in contact with at least two other gel bead of the plurality of closely packed gel beads. In some embodiments, a gel bead of the plurality of closely packed gel beads is in contact with at least three other gel bead of the plurality of closely packed gel beads.

In some embodiments, the plurality of gel beads comprises gel having a Young's modulus of 0.01 kPa to about 100 kPa. In some embodiments, the plurality of gel beads is buoyant in the first fluid stream. In some embodiments, the plurality of gel beads has a density of 800 kg/m3 to 1000 kg/m3.

In some embodiments, the gel outer layer comprises acrylamide. In some embodiments, the gel outer layer comprises agarose.

In some embodiments, the solid support is tagged using a molecular tag or barcode identifier such that contents of a tagged microfluidic droplet are identifiably mapped to a common source.

In some embodiments, the plurality of gel beads occupies greater than 30% of a volume of a segment of the first microfluidic channel. In some embodiments, the distance is less than a diameter of a gel bead of the plurality of gel beads.

In some embodiments, the method further comprises encapsulating the single solid support with a single cell in a droplet. In some embodiments, the method further comprises encapsulating the single solid support with cell lysis reagents in a droplet for performing cell lysis within the droplet. In some embodiments, the method further comprises combining the single solid support with reagents for nucleic acid synthesis in a droplet. In some embodiments, the method further comprises combining the single solid support with reagents for nucleic acid amplification in a droplet.

According to another aspect, a method of droplet generation can comprise: transporting a first fluid comprising a plurality of closely packed gel beads through a first microfluidic channel, a gel bead of the plurality of closely packed gel beads comprising a solid support and a gel outer layer encapsulating the solid support; and generating a plurality of droplets comprising a number of droplets containing a single gel bead, the generating comprising intersecting an immiscible carrier fluid and the first fluid by flowing the immiscible carrier fluid and the first fluid through a junction, and the plurality of droplets being generated substantially immediately after the junction, wherein the number of droplets is greater than 20% of the plurality of droplets.

In some embodiments, the number of droplets is greater than 50% of the plurality of droplets.

In some embodiments, a gel bead of the plurality of closely packed gel beads is in contact with at least two other gel bead of the plurality of closely packed gel beads.

In some embodiments, the solid support is tagged using a molecular tag or barcode identifier such that contents of a tagged microfluidic droplet are identifiably mapped to a common source.

In some embodiments, the gel outer layer comprises acrylamide. In some embodiments, the gel outer layer comprises agarose.

In some embodiments, the plurality of gel beads comprises gel having a Young's modulus of 0.01 kPa to about 100 kPa. In some embodiments, the plurality of gel beads is buoyant in the first fluid stream. In some embodiments, the plurality of gel beads has a density of 800 kg/m3 to 1000 kg/m3.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a schematic diagram of an example process for encapsulation of solid supports within a gel-precursor fluid to form of soft-gel beads comprising a solid support therein, in accordance with embodiments of the disclosure.

FIG. 2 is a micrograph illustrating an example of encapsulation of solid supports within a gel-precursor fluid to form soft-gel beads, where the soft-gel beads are flowed in a carrier oil, in accordance with embodiments of the disclosure.

FIG. 3 is a micrograph of an example of solid supports encapsulated within a polyacrylamide gel outer layer, after removal of the carrier oil, in accordance with embodiments of the disclosure.

FIG. 4 is a schematic diagram of a process for sorting gel beads by the number of solid supports contained therein, in accordance with embodiments of the disclosure.

FIG. 5 is a schematic diagram showing an example of regular spacing of gel beads comprising a single solid support therein, after reinjection into a microfluidic system, in accordance with embodiments of the disclosure.

FIG. 6 is a micrograph illustrating an example of reinjection of closely packed gel beads into a microfluidic device such that droplets comprising the gel beads are formed, in accordance with embodiments of the disclosure.

FIG. 7 is a micrograph illustrating an example of loading of gel beads into droplets, in accordance with embodiments of the disclosure.

FIG. 8A depicts soft particles packed at high density flowing into a single-file channel.

FIG. 8B depicts hard solid particles clumping in contact with solid surfaces in a microfluidic system.

### DETAILED DESCRIPTION

Disclosed herein are methods, compositions and systems relating to controllably generating in a microfluidic device a plurality of droplets which comprise a desired number of solid supports therein without any sorting of the droplets. Generation of droplets comprising the desired number of solid supports therein, such as a single solid support, can be directly achieved using droplet generation, without any droplet sorting steps. Solid supports can comprise any number of materials which can deliver reagents to a subsequent chemical reaction, including a chemical reaction within the microfluidic device downstream of the droplet generation. The chemical reaction can be any number of chemical reactions. In some instances, the chemical reaction comprises a nucleic acid synthesis reaction, including a nucleic acid synthesis reaction. For example, the reaction can comprise reverse transcription of RNA and/or DNA synthesis, such as single strand synthesis or amplification. In some cases, the solid supports can be used to deliver labels used to identify products of the synthesis and/or amplification reactions.

Solid supports are encapsulated within an outer gel layer. For example, one or more methods described herein relates to generation of a plurality of droplets comprising a gel bead therein, the gel bead comprising a solid support encapsulated within an outer gel layer. The outer gel layer can be compressible and/or have a density to facilitate transport of the gel bead through a microfluidic channel. For example, an outer gel layer can be selected to provide desired buoyancy for the gel bead to facilitate transport of the gel bead within the microfluidic channel. Encapsulation of a solid support within an outer gel layer can advantageously facilitate controllably spacing solid supports from one another within a microfluidic device, thereby facilitating increased efficiency in forming droplets containing a solid support therein. For example, solid supports encapsulated within an outer gel layer can be closely packed within a microfluidic channel without or substantially without clogging the microfluidic channel such that the solid supports encapsulated within an outer gel layer can be predictably flowed through the microfluidic channel, thereby enabling controlled generation of the droplets comprising the solid supports. Controllably spacing the solid supports close to one another within the microfluidic channel can facilitate controlled increase in generation of droplets comprising the solid supports. Increased efficiency in generating droplets comprising the solid supports can facilitate reduced waste and reduced time to generate a predetermined number of droplets, thereby reducing operating costs.

Disclosed herein are methods, compositions and systems relating to the generation of labeled droplets wherein the labeling demonstrates a non-Poissonian distribution. Also disclosed are droplet populations generated thereby, such that the droplet populations demonstrate labeling at a frequency that does not match a Poisson distribution, as well as reagents suitable for attaining such non-Poisson distributions. In particular, disclosed herein are labeled beads comprising a solid labeled core to which is added a compressible gel layer, such that the labeled beads possess at least one of the following properties: a buoyancy that, alone or in combination with the solid particle core, approximates that of a fluid medium through which droplets for which labeling is intended are suspended, transported and/or separated; a reversible compressibility, such that labeled beads held in proximity to one another and/or a will reversibly compress, so as to facilitate passage through a channel even when densely packed, without forming lightly packed aggregates. Such beads facilitate delivery of a population of labels to a population of droplets, and the sorting of a labeled droplet population, such that the labeled droplets demonstrate a distribution that is not a Poisson distribution.

Solid supports or solid particles, such as solid supports comprising one or more surfaces coated with oligonucleotides configured for use in single-cell barcoding, are often difficult to manipulate microfluidically. The rigid solid supports may have a high elastic modulus. As described in further details herein, the solid support may be made of various materials. In some embodiments, the solid support may comprise a polymeric material, including one or more of poly(methyl methacrylate), polycarbonate, and polystyrene. In some embodiments, the solid support can comprise silica. In some embodiments, the solid support can comprise a metal, including one or more of aluminum and steel. The elastic modulus of the solid support can depend on its compositions. For example, the solid supports as envisioned herein can have an elastic modulus, such as a Young's modulus, between about 0.5 GPa to about 200 GPa.

Such solid supports may be difficult to manipulate microfluidically due to their rigidity and/or tendency to jam in the channels of microfluidic devices, for example when the solid supports are loaded into the microfluidic devices at high concentrations. For example, solid supports suited for delivery of reagents may be difficult to manipulate microfluidically. Rigid solid supports flowed through channels of a microfluidic device may become lodged against a surface of a channel, such as when higher concentrations of the solid supports are flowed through the device, resulting in obstruction of the flow. Solid supports may have lower than desired buoyancy as compare to the fluid in which the solid supports are carried, or the carrier fluid. Solid supports having insufficient buoyancy as compared to the carrier fluid may sink to the bottom of the carrier fluid stream, thereby interfering with the carrier fluid stream flow within the channel. This jamming often makes pairing of the solid supports with one or more other components (e.g. droplets and/or cells) using a microfluidic device unreliable. For example, the solid support arrival times at a location within a microfluidic device for low concentrations may be governed by Poisson statistics, thereby leading to unpaired or overly paired components. In some embodiments, this disclosure relates to the encapsulation of a rigid or substantially rigid solid support within a gel to form a soft-gel bead or a gel bead. For example, a soft-gel bead may comprise a solid support core surrounded by a gel outer layer. Encapsulating solid supports with an outer gel layer can advantageously simplify the microfluidic handling of the solid supports. Encapsulating the solid supports can advantageously provide soft-gel beads which are deformable, and/or soft-gel beads which can be selected using optical detection and/or magnetic attraction techniques.

Deformable soft-gel beads may be less likely to jam within channels of microfluidic devices, thereby allowing loading of the soft-gel beads within the channels at higher concentrations Deformability of the soft-gel beads may facilitate movement of the beads around one another within the microfluidic device and/or movement of the beads along a portion of one or more surfaces of the channels. In some embodiments, the soft-gel beads may be densely packed within a channel of a microfluidic device without or substantially without clogging the channel. For example, soft-gel beads may be densely packed in a channel such that a soft-gel bead is in contact with one or more other soft-gel beads, and the beads can be moved within and/or through the densely packed channel without clogging the channel.

In some embodiments, a soft-gel bead comprising a solid support therein may demonstrate increased buoyancy as compared to the solid support alone. The gel outer layer may have a density lower than that of the solid support, and can be used for providing a soft-gel bead with desired buoyancy. For example, the gel outer layer may be selected such that the resulting soft-gel bead can have a desired density, thereby providing a soft-gel bead with desired buoyancy in the carrier fluid. In some embodiments, use of the outer gel layer can facilitate use of solid supports having increased density. For example, a gel outer layer may be selected based at least one its density such that the resulting soft-gel bead comprising the solid support and gel outer layer can maintain a desired density. In some embodiments, the gel outer layer may provide protection for a more fragile solid support. In some cases, the gel beads can have a density of about 500 kilograms per cubic meter (kg/m³) to about 1000 kg/m³, about 600 kg/m³ to about 1000 kg/m³, about 700 kg/m³ to about 1000 kg/m³, about 800 kg/m³ to about 1000 kg/m³, or about 900 kg/m³ to about 1000 kg/m³.

Use of soft-gel beads may enable selection of the content of the soft-gel beads, such as by using optical detection and/or magnetic attraction techniques. Combined with systems such as commercial cell sorters (e.g., fluorescence-activated cell sorting, FACS), for instance, the soft-gel beads can be rapidly sorted with ease to keep singly-loaded soft-gel beads, and to discard empty beads and/or those with more than one occupant. In some embodiments, densely packing soft-gel beads within a channel of a microfluidic device and use of soft-gel beads comprising a single rigid solid support can facilitate design of workflows in which the soft-gel beads can be introduced microfluidically at regular intervals such that each solid support can be reliably paired with one or more other components.

“Solid supports” and “solid particles” are used interchangeably herein to refer to rigid or substantially rigid physical structures comprising one or more surfaces upon which one or more tags or labels can be positioned.

“Soft-gel beads” and “gel beads” are used interchangeably herein to refer to a bead comprising a solid support or particle encapsulated within a gel outer layer.

A “nucleic acid molecule” or “nucleic acid” as referred to herein refers to deoxyribonucleic acid (DNA) or ribonucleic acid (RNA) including known analogs or a combination thereof unless otherwise indicated. Nucleic acid molecules to be profiled herein are variously obtained from any source of nucleic acid. The nucleic acid molecule is alternately single-stranded or double-stranded. In some cases, the nucleic acid molecule is DNA. Categories of DNA contemplated herein include mitochondrial DNA, cell-free DNA, complementary DNA (cDNA), DNA circulating in an individual's bloodstream, environmental sample DNA, synthetic DNA or genomic DNA. Often, the nucleic acid is genomic DNA (gDNA), such as DNA isolated from a healthy or diseased tissue from an individual. In some cases the genomic DNA comprises at least one structural mutation, such as a translocation, duplication, deletion or insertion, or at least one point mutation such as a SNP, that is distinctive, correlative or causative of aberrant cell behavior such as cancer. Categories of DNA include plasmid DNA, cosmid DNA, bacterial artificial chromosomes (BAC), or yeast artificial chromosomes (YAC). The DNA variously is derived from at least one chromosome, up to a complete diploid or polyploid chromosome set. For example, if the DNA is from a human, the DNA is derived from at least one of chromosome 1, 2, 3, 4, 5, 6, 7, 8, 9, 10, 11, 12, 13, 14, 15, 16, 17, 18, 19, 20, 21, 22, X, and Y.

The RNA includes, but is not limited to, mRNAs, tRNAs, snRNAs, rRNAs, retroviruses, small non-coding RNAs, microRNAs, polysomal RNAs, pre-mRNAs, intronic RNA, viral RNA, cell free RNA and fragments thereof. The non-coding RNA, or ncRNA can include snoRNAs, microRNAs (miRNAs), siRNAs, piRNAs and long nc RNAs.

The nucleic acid molecules are often contained within at least one biological cell. Alternately, the nucleic acid molecules are contained within a noncellular biological entity, such as, for example, a virus or viral particle. Nucleic acid molecules are often constituents of a lysate of a biological cell or entity. Nucleic acid molecules are often profiled within a single biological cell or a single biological entity. Alternately, nucleic acid molecules are profiled in a lysate obtained from a single biological cell or a single biological entity. The source of nucleic acid for use in the methods and compositions described herein are often a sample comprising the nucleic acid.

The term “barcode” refers to a known nucleic acid sequence that allows some feature of a nucleic acid (e.g., oligo) with which the barcode is associated to be identified. A barcode sequence is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 bases. A barcode sequence is at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 bases. A barcode sequence is about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 bases. An oligonucleotide (e.g., primer or adapter) comprises about, more than, less than, or at least 1, 2, 3, 4, 5, 6, 7, 8, 9, or 10 different barcodes. barcodes can be of sufficient length and comprise sequences that are sufficiently different to allow the identification of biological molecules based on barcode(s) with which each biological molecule is associated.

The term “oligonucleotide” as used herein refers to a nucleotide chain, typically less than 200 residues long, e.g., between 15 and 100 nucleotides long. The oligonucleotide comprises at least or about 1, 2, 3, 4, 5, 6, 7, 8, 9, 10, 15, 20, 25, 30, 35, 40, 45, or 50 bases. The oligonucleotides are from about 3 to about 5 bases, from about 1 to about 50 bases, from about 8 to about 12 bases, from about 15 to about 25 bases, from about 25 to about 35 bases, from about 35 to about 45 bases, or from about 45 to about 55 bases. The oligonucleotide (also referred to as “oligo”) is any type of oligo (e.g., primer). The oligonucleotides optionally comprise cleavable linkages. Cleavable linkages are optionally enzymatically cleavable. Oligonucleotides are single- or double-stranded. The term “primer” refers to an oligonucleotide capable of hybridizing to a complementary nucleotide sequence (e.g., the primer contains a nucleotide sequence that is complementary to a nucleotide sequence on a nucleic acid molecule). The term “oligonucleotide” can be used interchangeably with the terms “primer,” “adapter,” and “probe.”

The term “hybridization”! “hybridizing” and “annealing” can be used interchangeably and refer to the pairing of complementary nucleic acids.

The terms “polypeptide” and “protein” are sometimes used interchangeably herein to refer to polymers of amino acids of any length joined by peptide bonds. A polypeptide refers variously to any protein, peptide, protein fragment or component thereof. Some polypeptides are proteins naturally occurring in nature, while in other cases the term refers to a protein that is ordinarily not found in nature or that is synthesized. A polypeptide often consists largely of the standard twenty protein-building amino acids, but may be modified or synthesized to incorporate non-standard amino acids. A polypeptide is not uncommonly modified, typically by a host cell, by e.g., adding any number of biochemical functional groups, including phosphorylation, acetylation, acylation, formylation, alkylation, methylation, lipid addition (e.g., palmitoylation, myristoylation, prenylation, etc) and carbohydrate addition (e.g., N-linked and O-linked glycosylation, etc). Polypeptides often undergo structural changes in the host cell such as the formation of disulfide bridges or proteolytic cleavage.

As used herein, the term “about” a number refers to a range spanning that number plus or minus 10% of that number. The term “about” a range refers to that range minus 10% of its lowest value and plus 10% of its greatest value.

As used herein, the term “comparable to” a number refers to that number plus or minus 50% of that number. The term “comparable to” a range refers to that range minus 50% of its lowest value and plus 50% of its greatest value.

### Methods of the Disclosure

Disclosed herein are compositions, methods of forming and formulations related to the encapsulation of solid supports within a gel to form soft-gel beads. Also disclosed herein are various approaches for the use of such soft-gel beads, including for the delivery of at least one tag or label to a target entity. The solid supports can serve as supports to provide reagents for a chemical or biochemical assay. Generally, the methods disclosed herein include the encapsulation of rigid solid supports in gel to form deformable soft-gel beads, and the use of such gel encapsulated solid supports made thereby. Formation of the soft-gel beads and subsequent use of the soft-gel beads in delivery of a tag or label may be performed in a microfluidic device and/or system. In some embodiments, formation of the soft-gel beads and subsequent use of the soft-gel beads can be performed in the same microfluidic device.

Some methods of the disclosure involve flowing or moving a solid support or solid particle through or in a gel-precursor fluid and forming a droplet comprising the solid support surrounded by or encased by the gel-precursor fluid. The terms “drop,” “droplet,” and “microdroplet” are used interchangeably herein and refer to discrete entities comprising an aqueous phase and one or more components encapsulated in the aqueous phase, and can have a longest dimension, such as a diameter, ranging from about 0.1 μm to about 1000 μm. In some embodiments, a droplet can be produced in, on, or by a microfluidics device. In some embodiments, the gel-precursor fluid may comprise the aqueous phase, for example comprising water. In some embodiments, the droplet may be formed within an immiscible carrier fluid, the droplet comprising the solid support encapsulated within the gel-precursor fluid outer layer. The droplet may have a core comprising the solid support, and an outer layer comprising the gel-precursor fluid surrounding the inner core.

The solid supports can be spherical or substantially spherical. In some embodiments, solid supports are hollow. The solid supports can comprise an inner core comprising a space and an outer shell. In some embodiments, the solid supports are not hollow. In some embodiments, solid supports can be microbeads and/or microspheres. A number of solid particle compositions are consistent with the present disclosure. In some embodiments, a solid support can comprise poly(methyl methacrylate) (PMMA), polystyrene, polyethylene, polypropylene, silica (e.g., glass), metal, combinations thereof, and/or the like. In some embodiments, a solid support can comprise ceramic microspheres. In some embodiments, the solid supports can be made by curing a process. For example, the solid supports can be made of a polymer material formed by curing a precursor, such as epoxy precursors and/or ultraviolet curable polymers. In some embodiments, a solid support can comprise a magnetic material. For example, the solid supports may be made of a magnetic material to enable sorting of the solid supports using a magnetic attraction technique.

As described herein, in some cases, methods of fabricating and/or use of gel beads comprising solid supports comprises flowing the solid supports through one or more channels of a microfluidic device. The solid supports may be of a size suited for use within such microfluidic devices, for example being sized for desired flow through one or more channels of the microfluidic devices. A longest dimension of each of the solid supports may be selected such that the solid supports and/or the gel-bead formed therefrom can be moved through one or more channels of a microfluidic device. In some embodiments, a longest dimension of each of the solid supports can be less than a shortest dimension of the one or more channels. For example, the solid supports can each have a diameter smaller than the smallest diameter of the one or more channels through which the solid supports are configured to flow. In some embodiments, solid supports may have a diameter ranging from about 0.5 μm to about 200 μm. For example, a solid support may have a diameter of about 0.5 μm, about 0.6 μm, about 0.7 μm, about 0.8 μm, about 0.9 μm, about 1.0 μm, about 1.5 μm, about 2.0 μm, about 2.5 μm, about 3.0 μm, about 3.5 μm, about 4.0 μm, about 4.5 μm, about 5.0 μm, about 5.5 μm, about 6.0 μm, about 6.5 μm, about 7.0 μm, about 7.5 μm, about 8.0 μm, about 8.5 μm, about 9.0 μm, about 9.5 μm, about 10.0 μm, about 20 μm, about 30 μm, about 40 μm, about 50 μm, about 60 μm, about 70 μm, about 80 μm, about 90 μm, about 100 μm, about 120 μm, about 140 μm, about 160 μm, about 180 μm, about 200 μm or greater than about 200 μm. In some embodiments, solid supports can have a diameter of about 1 μm to about 200 μm, about 10 μm to about 70 μm, about 20 μm to about 60 μm, about 30 μm to about 50 μm, or about 40 μm. In some embodiments, solid supports can have a diameter of about 0.5 μm, about 0.6 μm, about 0.7 μm, about 0.8 μm, about 0.9 μm, about 1.0 μm, about 1.5 μm, about 2.0 μm, about 2.5 μm, about 3.0 μm, about 3.5 μm, about 4.0 μm, about 4.5 μm, about 5.0 μm, about 5.5 μm, about 6.0 μm, about 6.5 μm, about 7.0 μm, about 7.5 μm, about 8.0 μm, about 8.5 μm, about 9.0 μm, about 9.5 μm, about 10.0 μm, about 20 μm, about 30 μm, about 40 μm, about 50 μm, about 60 μm, about 70 μm, about 80 μm, about 90 μm, about 100 μm, about 120 μm, about 140 μm, about 160 μm, about 180 μm, about 200 μm or greater than about 200 μm. Exemplary diameters range from about 1 μm to about 200 μm, about 10 μm to about 70 μm, about 20 μm to about 60 μm, about 30 μm to about 50 μm, or about 40 μm.

One or more surfaces of the solid supports may be functionalized with one or more chemical moieties. Non-limiting examples of functional groups on one or more surfaces of the solid supports can include: alkyl, alkenyl, alkynyl, phenyl, halo, fluoro, chloro, bromo, iodo, hydroxyl, carbonyl, aldehyde, haloformyl, carbonate ester, carboxylate, carboxyl, ester, methoxy, hydroperoxy, peroxy, ether, hemiacetal, hemiketal, acetal, ketal, orthoester, methylenedioxy, orthocarbonate ester, carboxamide, primary amine, secondary amine, tertiary amine, quarternary amine, primary ketimine, secondary ketimine, primary aldimine, secondary aldimine, imide, azide, azo, cyanate, isocyanate, nitrate, nitrile, isonitrile, nitrosooxy, nitro, nitroso, oxime, pyridyl, sulfhydryl, sulfide, disulfide, sulfinyl, sulfonyl, sulfino, sulfo, thiocyanate, isothiocyanate, carbonothioyl, phosphino, phosphono, phosphate, borono, boronate, borino, borinate, combinations thereof, and/or the like. In some embodiments, one or more surfaces of the solid supports are functionalized to aid in binding and/or coating of the surfaces by one or more reagents. One or more surfaces of the solid supports may be functionalized to allow a chemical reaction to take place on those surfaces. In some embodiments, all or substantially all outwardly facing surfaces of a solid support are functionalized with one or more groups to facilitate binding of reagents to the surfaces.

Alternately or in combination, one or more surfaces of the solid supports can comprise one or more coating reagents. Coating reagents can include one or more molecular tags. In some embodiments, a molecular tag can include a protein, oligonucleotide, fluorophore, combinations thereof, and/or the like. In some embodiments, a molecular tag comprises a barcode identifier. In some examples, a solid support can comprise at least one type of molecular tag, such as a bar code identifier, configured to be used to identify that solid support from a population of solid supports. For example, the at least one type of molecular tag may have sufficient specificity such that the molecular tag may subsequently be used to tag one or more target nucleic acid components to identify the nucleic acid component as having a common source as the solid support. In some embodiments, the molecular tag on a solid support may be unique or sufficiently unique to that solid support such that the molecular tag can be used to tag a target to identify the target as having a common source as the solid support, including being from the same microdroplet.

In some embodiments, one or more surfaces of solid supports are coated with at least one oligonucleotide comprising a series of bases having a sequence that functions as barcode identifier configured to identify the corresponding solid support. Each of such barcode identifiers on a solid support may have a similar or same sequence of nucleotides such that the barcode identifiers can be grouped based on which solid support the identifiers are from after the barcode identifiers are removed from the solid support. In some embodiments, the corresponding barcode identifiers for each solid support are unique or sufficiently unique to that solid support within a population of solid supports, for example the pattern of the barcode identifiers occurring only once in the population of solid supports. Barcode identifiers may be configured to contain sufficient information to allow commonly tagged targets to be confidently mapped to a single source. For example, solid supports can be coated by at least one population of oligonucleotide primers, such as a population of oligonucleotide primers comprising a barcode identifier. The oligonucleotide primers may comprise a nucleic acid sequence that is complementary to a nucleic acid sequence on a target molecule such that the primers can hybridize to the nucleic acid sequence to identify the target molecule with the barcode identifier and allow tracing of the target molecule to the source in common with the solid support.

Alternately, one or more surfaces of a solid support may comprise heterogeneous populations of molecular tags that, in combination, convey sufficient information to allow commonly tagged targets to be confidently mapped to a single source, such as when individual identifiers do not comprise sufficient information for such mapping.

Methods of fabricating a gel bead provided herein can involve flowing or moving a solid support in or through a gel-precursor fluid and forming droplets comprising the solid supports within a gel-precursor fluid outer layer. A “gel-precursor fluid” is a liquid formulation configured to form a gel or gel-like substance after exposure to one or more stimuli. The gel-precursor fluid may comprise one or more polymers and/or polymer precursors. In some embodiments, the gel-precursor fluid can comprise a hydrogel. In some embodiments, the gel-precursor fluid comprises acrylamide, polyacrylamide and/or agarose. In some embodiments, the gel-precursor fluid can be an aqueous solution comprising acrylamide and/or agarose. Exposing the one or more polymers and/or polymer precursors to one or more stimuli may facilitate formation of the gel from the precursors. For example, exposure to the stimuli may induce polymerization reactions, including formation of cross-links between polymer chains to thereby facilitate formation of the gel from the gel-precursor fluid. For example, a gel-precursor fluid comprising acrylamide can be exposed to one or more stimuli to form a gel-bead comprising polyacrylamide. In some embodiments, exposure to one or more stimuli may induce solidification of the gel-precursor fluid. For example, agarose in a gel-precursor fluid may solidify to form an agarose gel when the temperature of the gel-precursor fluid is reduced.

Methods described herein can include transporting a plurality of controllably spaced gel beads in a first fluid through a first microfluidic channel and flowing an immiscible carrier fluid in a second microfluidic channel such that the first fluid and the immiscible carrier fluid intersect. The first microfluidic channel and the second microfluidic channel can join at a junction such that the first fluid and the immiscible carrier fluid can intersect to reliably generate a plurality of droplets comprising a gel bead encapsulated in the first fluid. The immiscible carrier fluid can segment the first fluid to generate the plurality of droplets. For example, the plurality of droplets can be generated immediately or substantially immediately after the junction of the first microfluidic channel and the second microfluidic channel. The gel bead can comprise a single solid support encapsulated within an outer gel layer such that the plurality of droplets comprise a number of droplets containing therein one gel bead comprising a single solid support encapsulated within an outer gel layer. For example, the number of droplets containing the gel bead comprising the single solid support can be generated immediately or substantially immediately after the intersection of the first fluid and the immiscible carrier fluid. The number of droplets can be generated without any sorting steps (e.g., without any sorting steps to selectively remove droplets containing a predetermined number of solid supports, such as removal of droplets containing more or less than one solid support, including droplets without a solid support and droplets with two solid supports). In some cases, the number of droplets containing a single gel bead comprising the single solid support can be greater than about 20%, about 30%, about 40%, about 50%, about 60%, about 70%, about 80% or about 90% of the plurality of droplets generated. The number of droplets generated as a proportion of the plurality of droplets can be greater than expected without controlled spacing between the gel beads in the first microfluidic channel, for example as compared to where spacing between the gel beads is random. In some cases, the number of droplets generated can be a greater than would be expected in a Poisson distribution. Methods described herein can include distributing a population of solid supports to a population of droplets such that a majority of the population of droplets receives a single solid support per droplet.

As described herein, the outer gel layer facilitates controlled spacing of the solid supports within the first microfluidic channel. The solid supports can be regularly spaced, enabling controllably positioning the solid supports at a predetermined distance relative to one another, within the first microfluidic channel. For example, the outer gel layer can facilitate closely packing the solid supports encapsulated in the outer gel layer, thereby enabling reliably increasing the rate at which the solid supports can be transported through the first microfluidic channel to generate droplets containing the solid supports. In some cases, a gel bead in the first microfluidic channel can be in direct contact with at least one, at least two, at least three, at least four, at least five, at least six, at least seven, or more other gel beads. In some cases, a gel bead can be at a distance of less than a longest dimension (e.g., diameter) from the nearest gel bead, including less than about 75%, about 50%, about 40%, about 30%, about 20% or about 10%, of the longest dimension from the nearest gel bead. In some cases, at least a segment of the first microfluidic channel is occupied by a composition having at least about 30%, at least about 40%, at least about 50%, at least about 60% or at least about 70%, of its volume occupied by gel beads comprising solid supports encapsulated by outer gel layers.

Controllably generating droplets containing a gel bead therein can facilitate controlled combination of the gel bead with one or more components downstream. For example, at least one component can be added to the droplets for a downstream reaction, including at least one component for a nucleic acid synthesis, such as a nucleic acid amplification process. In some cases, the one or more components comprise cells. In some cases, the one or more components comprise cell lysis reagents. Controllably generating droplets containing a gel bead therein facilitates increased efficiency in combining downstream components with gel beads, and thereby combination of the components with the solid supports. Methods described herein can facilitate increased efficiency in combining the droplets with cells, including pairing a single cell with a droplet containing a single gel bead comprising single solid support encapsulated within an outer gel layer. The pairing of the single cell with the droplet can be performed at a rate greater than would be expected without controlled generation of droplets containing a gel bead. Increased reliability of pairing the single cell with the droplet can increase process efficiency, reduce waste, and decrease operating costs.

In some cases, the first fluid comprises one or more reagents for a subsequent reaction. For example, the plurality of droplets generated by intersecting the first fluid and the immiscible carrier fluid can comprise a gel bead and the one or more reagents. In some cases, the first fluid does not contain reagents for a subsequent reaction.

Size of droplets generated can be controlled by adding fluid at or proximate to the junction between the first and second microfluidic channels. Fluid can be added to generate larger droplets. In some cases, the larger droplets can contain a larger quantity of reagents for a subsequent reaction. For example, the fluid added can include additional reagents. The added fluid can have the same or similar composition as the first fluid. In some cases, the added fluid has a different composition. For example, the gel beads can be controllably positioned within the first microfluidic channel to facilitate reliable generation of droplets containing the gel beads and fluid can be added at or proximate to the junction to controllably add predetermined reagents to the droplets and/or control the size of the droplets.

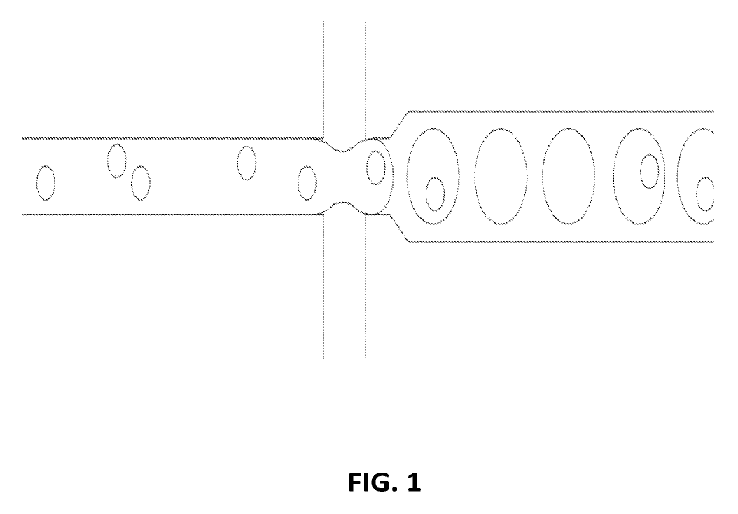

Referring to FIG. 1, a schematic diagram is shown of an example of distributing a plurality of solid supports within a plurality of fluid droplets. The plurality of solid supports can be randomly spaced relative to one another within the first microfluidic flow channel. As shown in the left portion of FIG. 1, the solid supports can be generally spaced far apart from one another while being transported in a first fluid within the first microfluidic flow channel. For example, a solid support can be at a distance of least three times a diameter of a solid support away from the nearest solid support, including at least four times, at least five times, at least 10 times or greater. The solid supports which are generally spaced far apart from one another can be transported through the first microfluidic flow channel without clogging the first microfluidic flow channel. An immiscible carrier fluid can be flowed through a second microfluidic flow channel such that the immiscible carrier fluid intersects with the first fluid transporting the solid supports to generate a plurality of droplets. In some cases, the second microfluidic flow channel flows a fluid of the same type as that in the first microfluidic flow channel for segmenting the fluid of the first microfluidic channel (e.g., not a fluid immiscible with the fluid in the first microfluidic flow channel). The plurality of droplets can then be transported away from the intersection of the first microfluidic flow channel and the second microfluidic flow channel in a third microfluidic flow channel. As shown in FIG. 1, some of the plurality of droplets can contain a solid support encapsulated in the first fluid. Some of the droplets can contain no solid supports. In some cases, some of the droplets can contain more than one solid support. An efficiency in generating droplets containing a desired number of solid supports can be negatively impacted by the irregular spacing between the solid supports in the first microfluidic flow channel. Irregular spacing between the solid supports in the first microfluidic channel can reduce the rate at which droplets containing a single solid support is generated, such as resulting in less than one or more than one solid support being encapsulated within one droplet. For example, the large distance between the solid supports in the first microfluidic channel can result in some droplets containing no solid supports.

FIG. 1 can illustrate an example of a process for encapsulating solid supports within a gel-precursor outer layer. As described herein, in some examples, the solid supports are flowed or transported through or in a gel-precursor fluid to form droplets comprising the gel-precursor fluid and the solid supports. Various droplet formation techniques may be used to form the one or more droplets comprising the gel-precursor fluid. In some embodiments, the droplets can be formed in a microfluidic device. FIG. 1 is a schematic depiction of a process for forming a droplet comprising a solid support within a gel precursor fluid outer layer. A population of solid supports can be introduced into a liquid or semiliquid continuous gel precursor flow. As described herein, the gel precursor may comprise one or more of agarose and acrylamide. In some embodiments, the gel-precursor fluid is hydrophilic. For example, the gel-precursor fluid may be an aqueous solution comprising a gel precursor (e.g., acrylamide or agarose). Solid supports may be transported through one or more channels of a microfluidic device in the continuous gel precursor stream. The solid support can be labeled as described herein, such as with one or more molecular tags. At left of FIG. 1, the small circles represent labeled solid supports. Solid supports consistent with the present disclosure can comprise any number of suitable materials, such as PMMA, polystyrene, methacrylate, silica, a metal, combinations thereof, and/or the like. Solid supports can be carried in the continuous gel-precursor fluid stream that is flowed through a first microfluidic flow channel of a microfluidic device.

The first microfluidic flow channel of the gel-precursor fluid stream can intersect and be in fluid communication with a second microfluidic flow channel of the microfluidic device. A carrier fluid stream immiscible with the gel-precursor fluid can be transported in the second microfluidic channel of the microfluidic device. In some embodiments, the immiscible carrier fluid flowed in the second microfluidic flow channel is hydrophobic. For example, the immiscible carrier fluid may be an immiscible carrier oil, such as a fluorocarbon oil. Forming the droplets comprising the gel-precursor fluid can comprise exuding the gel-precursor fluid from a flow endpoint of the first microfluidic flow channel, such as at the intersection of the first microfluidic flow channel with the second microfluidic flow channel. The gel precursor flow can be contacted with the carrier fluid stream at the intersection. The gel precursor can flow to the intersection of the channels such that individual gel precursor droplets bud off from the continuous flow to form droplets, at right. The gel-precursor fluid can be exuded from the first microfluidic flow channel at the intersection of the first microfluidic flow channel and the second microfluidic flow channel such that flow of the carrier fluid stream in the second microfluidic flow channel facilitates pinching off of a portion of the gel-precursor fluid, thereby forming a droplet comprising the gel-precursor fluid. Flow of the carrier fluid in the second microfluidic flow channel through the intersection can segment the flow of the gel precursor as the gel precursor travels through the intersection from a first portion of the first microfluidic flow channel to a second portion of the first microfluidic flow channel.

As shown in FIG. 1 in some embodiments, the first and third microfluidic flow channels may have openings on opposing portions of the second microfluidic flow channel such that the gel-precursor fluid may be exuded from the first microfluidic flow channel through the second microfluidic flow channel, and into the second portion of the first microfluidic flow channel. The droplet comprising the gel-precursor fluid can be subsequently flowed in the second portion of the first microfluidic flow channel within the microfluidic device in a carrier fluid. The carrier fluid flowed in the second microfluidic flow channel and the second portion of the first channel may be the same carrier fluid.

In some embodiments, the gel-precursor droplet may comprise a solid support. For example, a portion of the gel-precursor fluid segmented by the carrier fluid at the intersection of the first and second channels may comprise a solid support. In some embodiments, a gel-precursor droplet may comprise more than one solid support. In some embodiments, the gel precursor droplet may comprise no solid supports. As described in further detail herein, one or more sorting processes may be subsequently performed to provide a population of droplets comprising a desired number of solid supports. For example, a sorting process may be performed to provide a population of droplets where each droplet contains no more than one solid support.

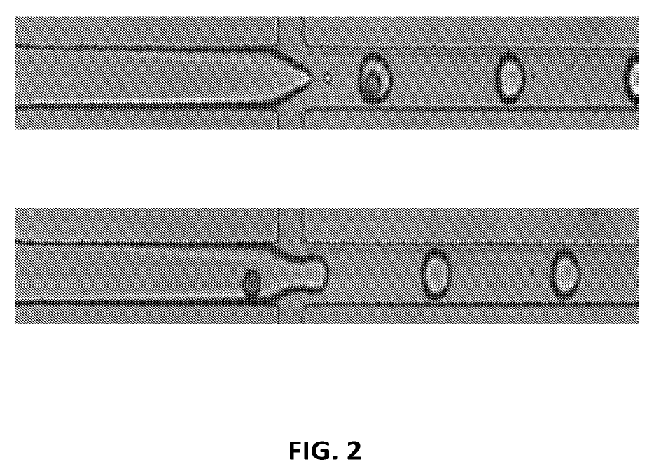

FIG. 2 shows micrographs of an example of distributing a plurality of solid supports within a plurality of fluid droplets. The process of FIG. 2 is similar to the process described with reference to FIG. 1. The solid supports of FIG. 2 can be spaced far apart from one another in a first channel. As shown in FIG. 2, a first fluid in the first channel can be intersected with an immiscible carrier fluid in a second microfluidic flow channel to generate droplets. In some cases, the droplets can contain one or more solid supports. In some cases, the droplets can contain no solid supports. For example, of the droplets shown in FIG. 2, some of the droplets contain no solid supports while one droplet contains one solid support. The increased spacing of the solid supports in the first channel can negatively impact the rate at which droplets containing one solid support are generated, thereby resulting in increased inefficiencies in the process.

The micrographs of FIG. 2 can be an example of a process for forming gel-precursor droplets. The micrographs of FIG. 2 show formation of droplets comprising a gel precursor fluid outer layer encapsulating a solid support therewithin. The micrographs demonstrate encapsulation of solid supports (dark beads, having a diameter of about 40 μm) into gel precursor droplets (lighter colored rounded structures, having a diameter of about 65 μm) in oil. A continuous stream of a gel precursor enters from the left, such as through a first portion of a first channel in a microfluidic device. The gel-precursor flow can comprise a low density flow of solid supports, for example as shown in the bottom micrograph of FIG. 2. The gel-precursor stream can intersect flow of an immiscible fluid (e.g., oil) in a second channel, for example shown at the center of the micrographs, thereby causing gel precursor droplets to bud off sequentially. Droplets containing one or more solid supports can be formed as the continuous flow of the gel precursor is segmented. The droplets can be transported in the immiscible fluid after formation. For example, the immiscible fluid can serve as a carrier fluid in the second portion of the first channel such that the gel-precursor droplets may be transported in the immiscible fluid in the second portion of the first channel after formation. Gel precursor droplets remain separate from one another in the immiscible fluid flow in the second portion of the first channel. The top micrograph of FIG. 2 shows formation of a droplet comprising a solid support core. At the bottom micrograph, it is shown that a second droplet comprising a solid support core will from in a subsequent gel budding.

As depicted in FIGS. 1 and 2, one or more of the droplets comprising the gel-precursor fluid can contain at least one solid support. In some embodiments, one or more of the gel-precursor fluid droplets does not contain a solid support. In this depiction, 60% (and, with the budding droplet, 66%) of the droplets comprise a gel precursor outer layer encapsulating a solid support. In some embodiments, one or more other components can be added to one or more of the gel-precursor droplets. In some embodiments, the one or more other components can be one or more of cells, proteins, and nucleic acids. For example, the one or more other components may comprise a target molecule.

In some embodiments, droplets comprising the gel-precursor fluid encapsulating one or more solid supports can be converted into gel droplets, or droplets comprising a gel or gel-like outer layer encapsulating one or more solid supports. For example, the droplets comprising the gel-precursor fluid may be converted to a gel beads comprising one or more solid supports encapsulated therein. The gel-precursor fluid, when subjected to one or more stimuli, may be converted from a liquid state to a gelatinous or gelatinous-like state. As describe herein, in some embodiments, the gel or gel-like outer layer may comprise a hydrogel, including a gel comprising polyacrylamide and/or agarose gel. In some embodiments, the gel or gel-like outer layer may consist or consist essentially of polyacrylamide. In some embodiments, the gel or gel-like outer layer may consist or consist essentially of agarose. In some embodiments, forming the gel outer layer comprises inducing cross-link formation of polyacrylamide. In some embodiments, forming the gel outer layer comprises inducing solidification of liquid agarose to form agarose gel.

In some embodiments, gel or gel-like outer layer may comprise a superabsorbent polymer. In some embodiments, the gel or gel-like outer layer may consist or consist essentially of a superabsorbent polymer. In some embodiments, a superabsorbent polymer can be formed by polymerization of acrylic acid blended with sodium hydroxide to form a poly-acrylic acid sodium salt, such as sodium polyacrylate. In some embodiments, a superabsorbent polymer can be made using one or more of a polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile.

Any stimulus, alone or in combination with one or more other stimuli, capable of converting the gel-precursor fluid into a gelatinous or gelatinous-like state is consistent with the disclosure herein. In some embodiments, the one or more stimuli may comprise one or more of a physical and chemical stimulus. A physical stimulus may comprise one or more of a temperature, electric field, magnetic field, light or pressure stimulus. A chemical stimulus may comprise exposing the gel-precursor fluid to one or more of a pH, an ionic strength, a solvent composition, and a molecular species stimulus. In some embodiments, a stimulus may be a change in temperature. For example, forming the gel or gel-like substance may comprise altering a temperature of the gel-precursor fluid (e.g., cooling the gel-precursor fluid). In some embodiments, forming the gel or gel-like substance may comprise crystallizing the gel-precursor fluid. Methods of converting the gel-precursor fluid into a gelatinous or gelatinous-like state consistent with the disclosure herein can comprise inducing polymerization and/or formation of cross-links. In some embodiments, the gel-precursor fluid may be exposed to one or more stimuli to induce formation of cross-linkage within and/or between polymers of the gel-precursor fluid. For example, acrylamide in a gel-precursor fluid may be subjected to one or more stimuli such that polymerization occurs to form a gel bead comprising polyacrylamide. In some embodiments, forming the gel or gel-like substance may comprise solidifying the gel-precursor fluid. For example, liquid agarose of a gel-precursor droplet may be cooled such that the liquid agarose can solidify to form gel beads comprising agarose.

In some embodiments, conversion of the gel-precursor fluid to a gel or gel-like substance is performed while the droplet comprising the gel-precursor fluid is within or surrounded by an immiscible carrier fluid. In some embodiments, conversion of the gel-precursor fluid to a gel or gel-like substance is performed after the solid support is encapsulated within the gel-precursor fluid such that the solid support is not in contact with the immiscible carrier fluid. A droplet comprising the gel-precursor fluid and solid support may be exposed to one or more stimuli for converting the gel-precursor fluid to a gel or gel-like substance while the droplet is within the immiscible carrier fluid. For example, conversion of the gel-precursor fluid to a gel can be performed subsequent to the steps described with reference to FIGS. 1 and 2. In some embodiments, gel beads may be removed from the carrier fluid subsequent to conversion of the gel-precursor fluid to the gel. In some embodiments, the gel beads may be separated from the carrier fluid to facilitate further processing of the gel beads. As described herein, the gel beads may be sorted subsequent to formation so as to provide an enriched population of gel beads comprising a desired number of solid supports. In some embodiments, removing the gel beads from the carrier fluid facilitates a more efficient enrichment process to provide the population of gel beads comprising the desired number of solid supports. In some embodiments, the gel beads may be subsequently sorted to provide an enriched population of gel beads comprising a single solid support. The gel beads of the enriched population may be reinjected into a fluid stream, such as an aqueous fluid stream, for further processing, including combination with one or more target entities.

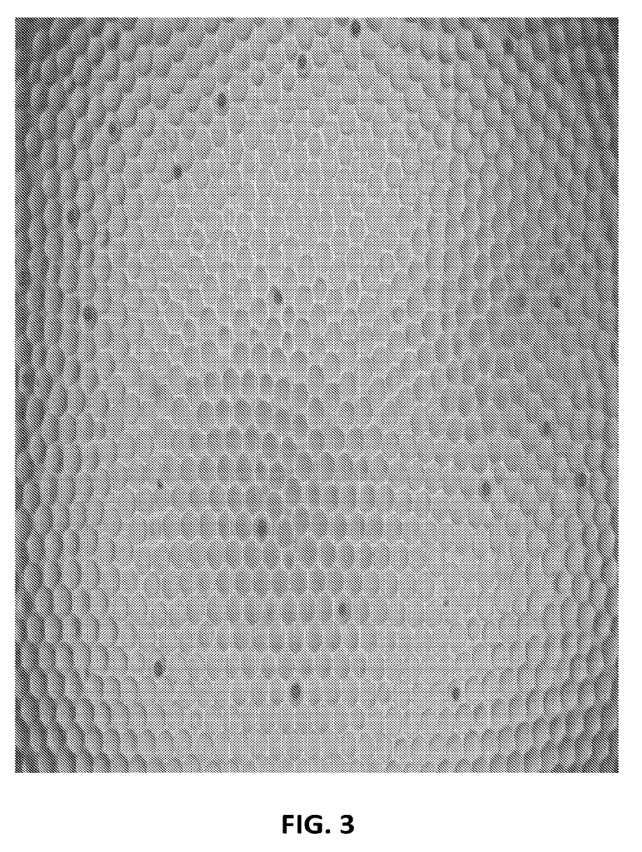

FIG. 3 is a micrograph showing a population of droplets generated using a process comprising steps described with reference to FIGS. 1 and 2. As shown in FIG. 3, some of the droplets contain a solid support encapsulated therein (e.g., shown as a droplet comprising a darker colored core). Some of the droplets do not contain any solid supports. Many of the droplets do not contain any solid supports, for example more droplets do not contain solid supports than those which do.

In an example, FIG. 3 is a micrograph of an example of a population of gel beads formed according to processes described with reference to FIGS. 1 and 2. Some of the gel beads in FIG. 3 comprise a single solid support core. The gel beads can be uniform or substantially uniform in volume. The gel beads of FIG. 3 comprise polyacrylamide gel droplets removed from the carrier fluid (e.g., carrier oil). The gel beads can be formed from gel-precursor droplets comprising acrylamide, as described herein. For example, droplets comprising an acrylamide gel precursor fluid may be subjected to conditions to allow polymerization of the acrylamide to form the polyacrylamide gel. Many of the gel beads do not contain a solid support therein. More gel beads do not contain any solid supports therein than those which do. As described herein, the reduced efficiency with which gel beads containing a solid support is formed can be due to irregular spacing between solid supports prior to the formation of the droplets, decreasing the ability to control the number of droplets which contain a solid support.

As described herein, the gel or gel-like bead may demonstrate a desired degree of deformability (e.g., a deformable bead or deformable particle). An object's resistance to being deformed elastically is measured by its elastic modulus. The elastic modulus can depend on a degree of cross-linkage formed in polymeric gel beads. The deformable beads such as gel beads as envisioned herein can have an elastic modulus, such as a Young's modulus, of about 0.01 kPa to about 100 kPa. In some embodiments, the elastic modulus of gel beads can be about 0.01 kPa to about 0.1 kPa, or about 0.1 kPa to about 1 kPa. In some embodiments, the elastic modulus of the gel beads can be about 1 kPa to about 10 kPa. For example, the deformable beads have an elastic modulus of about 0.5 kPa, about 1.0 kPa, about 1.5 kPa, about 2.0 kPa, about 2.5 kPa, about 3.0 kPa, about 3.5 kPa, about 4.0 kPa, about 4.5 kPa, about 5.0 kPa, about 5.5 kPa, about 6.0 kPa, about 6.5 kPa, about 7.0 kPa, about 7.5 kPa, about 8.0 kPa, about 8.5 kPa, about 9.0 kPa, about 9.5 kPa, about 10.0 kPa or greater than about 10.0 kPa. In some embodiments, the elastic modulus of the gel beads can be about 0.1 kPa to about 60 kPa, about 1 kPa to about 60 kPa, about 10 kPa to about 60 kPa, about 20 kPa to about 60 kPa, about 20 kPa to about 40 kPa. Sometimes, deformable beads can have an elastic modulus of 1-10 kPa. For example, the deformable beads can have an elastic modulus of 0.5 kPa, 1.0 kPa, 1.5 kPa, 2.0 kPa, 2.5 kPa, 3.0 kPa, 3.5 kPa, 4.0 kPa, 4.5 kPa, 5.0 kPa, 5.5 kPa, 6.0 kPa, 6.5 kPa, 7.0 kPa, 7.5 kPa, 8.0 kPa, 8.5 kPa, 9.0 kPa, 9.5 kPa, 10.0 kPa or greater than 10.0 kPa. The elastic modulus of the deformable bead can be less than the elastic modulus of a solid support contained therein (e.g., the solid support is more rigid than the deformable bead).

The gel or gel-like bead may be spherical or substantially spherical. In some embodiments, the gel or gel-like bead can have a size of about 1 μm to about 200 μm in a longest dimension, such as diameter, including about 1 μm to about 20 μm, about 10 μm to about 15 μm, about 20 μm to about 50 μm, about 35 μm to about 70 μm, about 50 μm to about 100 μm or about 100 μm to about 200 μm. For example, the deformable gel bead may have a size of about 1 μm, about 5 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 120 μm, about 140 μm, about 160 μm, about 180 μm, about 200 μm or greater than about 200 μm in diameter. In some embodiments, the gel or gel-like bead has a size of about 1 to about 200 μm in diameter, about 1 to about 20 μm in diameter, about 10 to about 15 μm in diameter, about 20 μm to about 50 μm in diameter, about 35 μm to about 70 μm in diameter, about 50 μm to about 100 μm or about 100 μm to about 200 μm in diameter. For example, the deformable gel bead has a size of about 5 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 120 μm, about 140 μm, about 160 μm, about 180 μm, about 200 μm or greater than about 200 μm in diameter.

The gel or gel-like bead may have a size that is greater than the solid support or solid particle contained therein, such that the solid support or solid particle is entirely or substantially entirely encapsulated by the deformable bead. For example, the deformable gel bead may have a size that is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100% or more than about 100% greater than the solid support or solid particle contained therein.

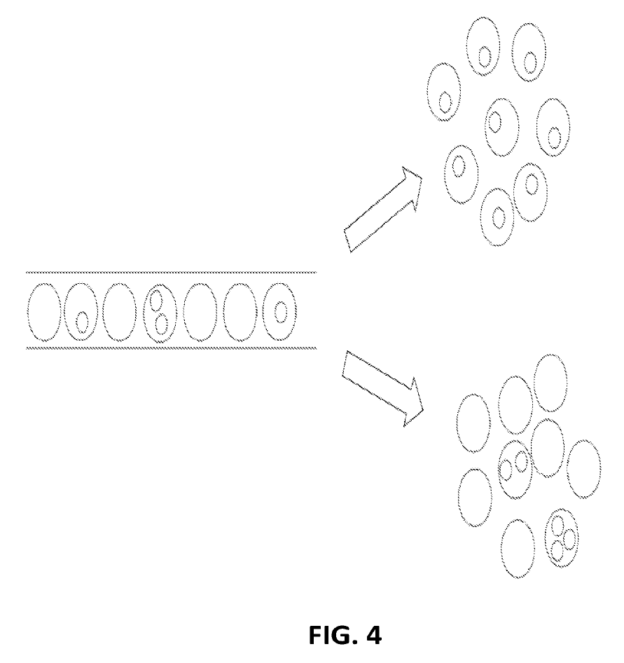

As described herein, a number of droplets which contain a predetermined number of solid supports (e.g., a single solid support) generated according to processes comprising the steps described with reference to FIGS. 1 and 2, can be at a proportion of the total number of droplets generated which is lower than desired. One or more sorting steps can be performed to provide an enriched population of droplet containing the predetermined number of solid supports. Referring to FIG. 4, a schematic diagram is shown of an example of a process for retaining the droplets with one solid support encapsulated therein. One or more sorting steps can be performed to selectively remove droplets which contain less than or more than the predetermined number of solid supports. Various sorting processes can be used, including at least one of sorting by mass, fluorescence, magnetic properties, and electrical properties. For example, a property of the solid support can be used for the sorting such that droplets with a solid support will indicate a positive for the presence and/or quantity of the solid support.

The methods provided herein optionally involve generating enriched populations of gel beads, wherein a majority of the gel beads contain a single solid support or solid particle. Generating enriched populations of gel beads, wherein a majority of gel beads contain a single solid support or solid particle, involves encapsulating solid supports or solid particles in a gel-precursor fluid substantially as described herein, converting the gel-precursor fluid to a gelatinous state, and selecting and/or sorting gel beads that contain a single solid support or solid particle. In some embodiments, the gel beads may be sorted to provide a population of gel beads where each bead comprises one or no solid support.

It may be desirable for each of the gel beads to contain no more than one solid support or solid particle to facilitate desired pairing of the solid support with one or more other components, such as a component comprising a target molecule. Gel beads containing the desired number of encapsulated solid supports can be selected for further use whereas those gel beads containing an undesired number of encapsulated solid supports can be discarded or reserved for a further use. The methods provided herein optionally can comprise sorting, grouping, clustering, and/or segregating the gel beads according to the number of solid supports or solid particles contained therein. The methods involve assaying for one or more properties of the solid supports or solid particles within the gel beads. Assaying for one or more properties can involve, but is not limited to, assaying for various physical and/or chemical properties.

In one example, FIG. 4 is a schematic diagram of a gel beads population being generated through sorting. As shown in FIG. 4, gel beads that contain a single solid support can be segregated from gel beads that contain more than or less than a single solid support. As described herein, gel beads can be extracted or removed from the immiscible carrier oil, such as shown in FIG. 3 (e.g., a plurality of solid supports encapsulated within polyacrylamide gel beads after removal of the carrier oil). In some embodiments, gel beads separated from the immiscible carrier oil can be resuspended, for example in an aqueous solution, to facilitate subsequent sorting, grouping, clustering, and/or segregating. An enriched population of gel beads comprising a desired number of solid supports can be provided through the sorting, grouping, clustering, and/or segregating. For example, a population of gel beads where each or substantially each of the gel beads comprising a single solid support or solid particle, can be provided.

In some embodiments, methods of the disclosure may comprise providing a population of gel beads where each gel bead of the population comprises no more than one solid support encapsulated therein through one or more of sorting steps. Beads are variously sorted by assaying for fluorescence, light absorption, magnetic properties, electrical properties, density of the solid support, buoyancy of the solid support, density of the labeling beads, buoyancy of the labeling beads, rigidity of the solid support, or other properties corresponding to solid support number within a bead. In some embodiments, a population of gel beads can be provided where each gel bead comprises one solid support encapsulated therein. In some embodiments, the methods comprise converting the gel-precursor fluid into gel or gel-like beads such that a majority of the gel or gel-like beads encapsulate no more than one solid support.

In one example, assaying involves measuring or detecting fluorescence of the solid support or particle contained within the gel bead. Fluorescence can be a property of the solid support itself (e.g., a fluorescent bead) and/or the solid support is coated with one or more fluorescent moieties (e.g., a fluorescent marker). A fluorescence intensity level of the gel beads may be detected wherein the fluorescence intensity level can depend on the number of solid supports contained therein. For example, an increased fluorescence intensity may be detected from gel beads comprising a higher number of solid supports. In this example, gel beads can be sorted or segregated based on the level of fluorescence, for example, by illuminating the gel beads with a laser and directing gel beads to one or more reservoirs according to the level of fluorescence detected.

Alternately or in combination, light absorption of the gel beads can be measured to facilitate sorting of the gel beads. For example, the amount of light absorbed by a gel bead can depend upon the number of solid supports or particles contained within the gel bead. Alternate methods of assaying for the number of solid supports contained within a gel bead can include, without limitation, detecting magnetic or electrical properties of the gel bead, and/or measuring a density and/or buoyancy of the gel beads containing a solid support.

Using these methods, a population of gel beads containing a single solid support are generated, wherein greater than about 50%, greater than about 55%, greater than about 60%, greater than about 65%, greater than about 70%, greater than about 75%, greater than about 80%, greater than about 85%, greater than about 90%, greater than about 95%, and up to 100% of the gel beads in the population contain a single solid support or solid particle. Enriched populations of gel beads are often generated, wherein each or substantially each of the gel beads in the enriched population contains a single solid support or particle.

For example, the methods may comprise converting the gel-precursor fluid into gel or gel-like beads such that at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least greater than about 99% of the gel or gel-like beads encapsulate no more than one solid support or solid particle. The methods involve converting the gel-precursor fluid into gel or gel-like beads such that at least about 10% to about 100%, at least about 10% to about 50%, at least about 20% to about 60%, at least about 30% to about 70%, at least about 30% to about 50%, at least about 40% to about 60%, or at least about 50% to about 80% of the gel or gel-like beads encapsulate no more than one solid support or solid particle.

In some embodiments, beads are sorted so as to generate a population having solid particles in beads at a frequency other than a Poisson distribution, such as at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or greater than 95% of the beads in at least one sorted population comprise one solid particle per bead.

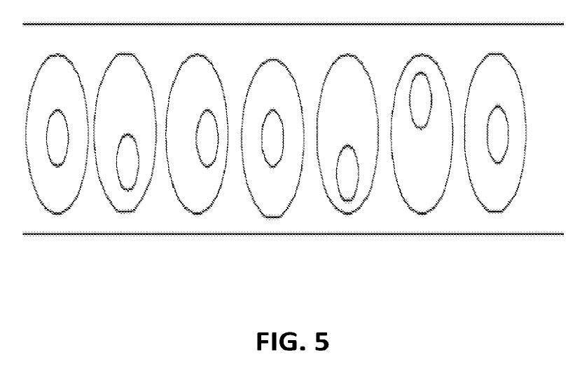

FIG. 5 is a schematic diagram of a population of droplets enriched for the desired number of solid supports within a droplet. For example, the population is enriched for droplets which contain a single solid support. The enriched population can be generated using a process comprising one or more sorting steps, such as sorting steps described with reference to FIG. 4. In some cases, a population comprising a predetermined number of droplets comprising a predetermined number of solid supports therein can be generated using one or more sorting steps described with reference to FIG. 4. In some cases, a uniform or substantially uniform droplet population comprising droplets containing predetermined number of solid supports therein can be generated.

In an example, FIG. 5 is a schematic diagram of an enriched population of gel beads where a predetermined number of gel beads contain a predetermined number of solid supports. The sorted gel beads can be reinjected into a microfluidic device, as depicted, for example, in FIG. 5. For example, gel beads each comprising a single solid support may be reinjected into a microfluidic device for pairing with one or more other components. The sorted gel beads may be transported within a channel of the microfluidic device with regular spacing. In some embodiments, the sorted, or enriched populations of gel beads, can be transported with regular spacing within a microfluidic channel, for example even when flowed at a high density.

Gel beads may be flowed in one or more channels of a microfluidic device so as to form droplets comprising the gel beads. In some embodiments, gel beads can be transported within the one or more channels in an aqueous carrier fluid such that aqueous droplets comprising the gel beads can be formed. For example, droplets comprising one or more gel beads encapsulated in an aqueous phase may be formed.

In some cases, an enriched population of droplets where the droplets contain a desired number of solid supports can be generated directly from droplet generation, in the absence of any sorting of the droplets. The population of droplets generated can directly comprise the desired number of solid supports within the droplets, without a step of sorting the droplets. The population of droplets can be generated using a population of gel beads comprising the desired number of solid supports encapsulated within compressible outer gel layers. For example, the population of gel beads can comprise a predetermined proportion of which comprises the desired number of solid supports encapsulated within a compressible outer gel layer. The population of gel beads can comprise a predetermined proportion of which comprises a single solid support within a compressible outer gel layer. The gel beads can be positioned at controlled distances relative to one another within a microfluidic flow channel. For example, controlled generation of the population of droplets can be achieved through the use of densely packing in a fluid flow the gel beads comprising the desired number of solid supports encapsulated therein. The compressible gel coating can facilitate tight packing of the solid supports without any clumping and/or clogging of a microfluidic flow channel. In some cases, a tightly packed gel bead in a microfluidic channel can be in direct contact with at least one, at least two, at least three, at least four, at least five, at least six, at least seven, at least eight, at least nine, at least ten or more other gel beads. In some cases, a closely packed gel bead can be at a distance of less than a longest dimension of the gel bead from the nearest gel bead. For example, the closely packed gel bead can be at a distance of less than a diameter of the gel bead from the nearest gel bead, including less than about 75%, about 50%, about 45%, about 40%, about 35%, about 30%, about 25%, about 20%, about 15% or about 10%, of the diameter from the nearest gel bead. In some cases, at least a segment of the microfluidic channel comprising the closely packed gel beads can comprise at least about 30%, at least about 35%, at least about 40%, at least about 45%, at least about 50%, at least about 55%, at least about 60%, at least about 65%, or at least about 70%, of its volume occupied by the gel beads.

An enriched population of gel beads comprising the predetermined proportion that comprises a desired number of solid supports encapsulated therein can be used to generate the enriched population of droplets. The enriched population of gel beads can be obtained through sorting of an initial population of gel beads. As described herein, gel beads comprising more or less solid supports than the desired number of solid supports can be removed using one or more sorting steps to provide an enriched population of gel beads where a predetermined proportion of the population comprises the desired number of solid supports. For example, the initial population of gel beads can be sorted to generate the enriched population of gel beads. Generating droplets which contain a desired number of solid supports therein using an enriched population of gel beads which contain the desired number of solid supports can improve process control, reduce or eliminate undesired reactions between reagents, reduce or eliminate degradation of reagents, and/or increase processing speed. Sorting of gel beads can be more easily performed than sorting of droplets. Droplets can more easily combine with one another and/or disintegrate during manipulation. Gel beads can be more robust and more easily manipulated. In some cases, sorting of gel beads can be performed earlier in a process, for example further upstream in the process flow, such that combination of reagents with the desired number of solid supports can be performed later, reducing the amount of time reagents are in the presence of each other and the solid supports prior to the reaction, and reducing the complexity of the process once reagents are combined with one another and/or the solid supports. Gel beads can be closely packed with one another while droplets may combine with one another when positioned in close proximity or in contact with one another.

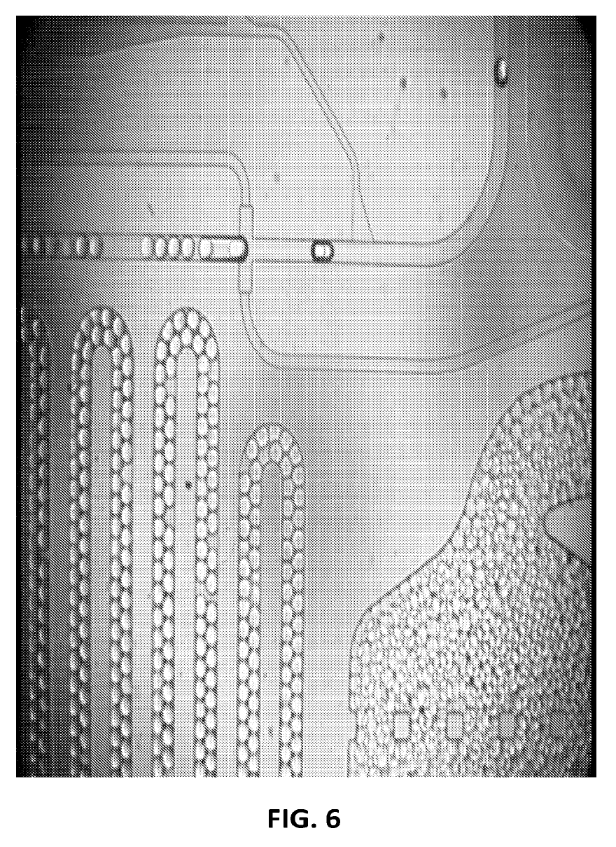

At FIG. 6 a micrograph is shown of densely packed gel beads flowing through a microfluidic system. At lower left, a large, multi-file population of gel particles flow en masse, frequently contacting one another without clumping or clogging the microfluidic system. At left, the gel beads are flowed into double-file channels, and are seen flowing at a regular, high density in the microfluidic flow channels. Individual gel beads are seen to be in physical contact with up to 5 or more adjacent gel beads, without disrupting microfluidic flow.

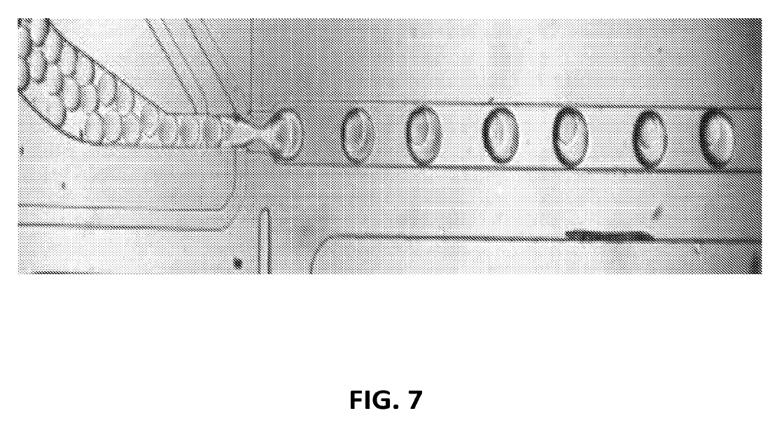

At FIG. 7, a micrograph is shown of the generation of a uniform or substantially uniform droplet population without any sorting steps to selectively remove droplets which do not contain a predetermined number of solid supports. At left, densely packed gel beads in a liquid flow toward a droplet generation junction. The gel beads are densely packed, with individual gel beads contacting multiple adjacent particles in the flow, without clogging or disrupting flow in the microfluidic channel. Following the droplet junction, center, an emulsion of individual droplets is generated. Droplets each comprise a single gel beads in a uniform or substantially uniform emulsion, immediately after droplet generation with no further sorting.

The gel beads used for droplet generation can be enriched for a desired number of solid supports. For example, the gel beads may be pre-sorted to obtain a population of gel beads comprising a proportion of which comprises a desired number of solid supports encapsulated therein. As described he

FIGS. 6 and 7 are micrographs of examples processes of gel beads being introduced into a microfluidic device, and encapsulating the gel beads into emulsion droplets. The gel beads may be formed as described herein. After extraction from the carrier oil and sorting, the gel beads may be introduced into an aqueous solution in a microfluidic device. The gel beads can be positioned at controlled distances relative to one another to facilitate controlled generation of droplets. As shown in FIGS. 6 and 7, the gel beads can initially be closely packed in the aqueous solution without clogging any of the channels of the microfluidic device. The gel beads may be densely packed such that the gel beads are in direct contact with adjacent gel beads, or in close proximity to adjacent gel beads. The gel beads may be densely packed such that the beads are in direct contact with one or more interior surfaces of one or more channels of the microfluidic device. Densely packing the gel beads can enable increased efficiency at which droplets are generated which contain a predetermined number of gel beads, such as a single gel bead. This may advantageously enable forming fluid droplets containing a desired number of solid supports without sorting generated droplets. Sorting fluid droplets may be complex, and the droplets difficult to manipulate. Controllably forming fluid droplets containing a desired number of solid supports directly through droplet generation eliminates the need to sort fluid droplets to obtain an enriched population of fluid droplets containing the desired number of solid supports. In some cases, closely packing gel beads may advantageously reduce the volume needed to process the gel beads, thereby facilitating a reduction in the size of the microfluidic device.

Forming droplets comprising the one or more gel beads can comprise transporting the gel beads in the aqueous fluid stream from the closely packed section through a first portion of a second flow channel of the microfluidic device, such as through the channels shown on the left in each of FIGS. 6 and 7. Each of the gel beads can be introduced into the first portion of the second channel at a desired interval to facilitate formation of the droplets. The second channel can intersect and be in fluid communication with a third channel of the microfluidic device carrying a fluid immiscible with the aqueous solution of the second channel. For example, an immiscible oil can be flowed in the third channel. The aqueous fluid stream can transport the gel bead through the first portion of the second channel to an endpoint of the first portion, such as at the intersection of the second and third channels. The aqueous solution and gel bead can contact the immiscible fluid in the third channel at the intersection. A droplet comprising an aqueous outer phase surrounding a solid support can be formed as the aqueous fluid stream is segmented by the immiscible fluid of the third channel in the intersection of the second and third channels. The droplets comprising the aqueous phase and one or more gel beads may be flowed in or through an immiscible carrier fluid, such as a carrier oil, in a second portion of the second channel. The aqueous fluid stream may or may not contain reagents for a subsequent reaction. In some cases, the aqueous fluid stream comprises reagents for a subsequent reaction such that the droplet formed comprises the reagents in the aqueous fluid, the aqueous fluid encapsulating the gel bead. In some cases, additional fluid, comprising reagents or not comprising reagents, can be added, at or adjacent to the intersection, to the aqueous fluid prior to formation of the droplets. Addition of the fluid can be used to control a droplet size and/or for providing additional reagents to the droplets that are generated. For example, as shown in FIG. 7, a fourth channel next to the third channel can be in fluid communication with the second channel to deliver additional fluid to the aqueous fluid in the second channel prior to forming the droplets.

A majority of the droplets can encapsulate a single gel bead (e.g., a “bead within a bead within a drop”). In cases where an enriched population of gel beads is utilized (e.g., a majority of the gel beads contain a desired number of encapsulated solid supports), at least 30%, at least 35%, at least 40%, at least 45%, at least 50%, at least 55%, at least 60%, at least 65%, at least 70%, at least 75%, at least 80%, or greater than 80% of the droplets contain a single solid support or particle.

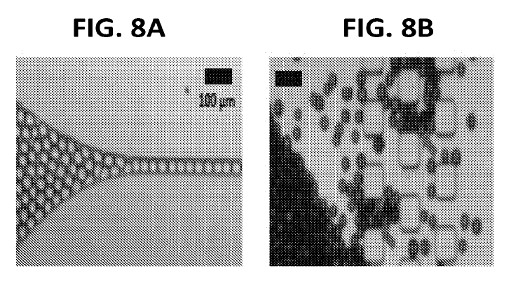

At FIG. 8A, a micrograph is shown of densely packed gel beads flowing through a narrowing microfluidic channel. Single gel beads contact multiple adjacent beads. The gel beads can compress and reform their uncompressed shapes in response to pressure from adjacent beads or channel walls.

At FIG. 8B, a micrograph is shown of solid supports, uncoated, passing through a microchannel landscape. At the positions indicated by the dark arrows, center and top, the particles are seen to clump in channels created by columns in the microfluidic landscape, despite the channels being substantially wider than the solid bead diameter At lower left, one sees a large clump of solid supports, withdrawn from the liquid flow and static in the microfluidic system. This contrasts to FIG. 8A, where gel beads maintaining flow capabilities despite being in frequent contact with one another and the channel walls, and despite flowing through substantially more narrow channel.

Methods of Pairing Gel Beads with Target Entities

Disclosed herein are methods for pairing gel beads with one or more target entities. The one or more target entities can be biological cells or lysates thereof. The methods can comprise pairing a single gel bead with a single biological cell or entity, or a cell lysate from a single biological cell or entity. The methods can comprise pairing a single gel bead with a single target entity within a single droplet. In some embodiments, a droplet may comprise a single cell, or lysate of a single cell, and a single gel bead. For example, the methods may comprise pairing a single gel bead with a single target entity in a droplet, such that at least about 30%, about 35%, about 40%, about 45%, about 50%, about 55%, about 60%, about 65%, about 70%, about 75%, about 80%, about 85% or greater than about 85% of the droplets contain a single gel bead and a single target entity. Put another way, the methods can comprise pairing a single gel bead with a single target entity in droplets, such that less than about 70%, about 65%, about 60%, about 55%, about 50%, about 45%, about 40%, about 35%, about 30%, about 25%, about 20%, about 15%, about 10%, about 5% or less than about 5% of the droplets are overpaired (e.g., the number of gel beads in a droplet is more than one and/or the number of target entities in a droplet is more than one) or underpaired (e.g., the number of gel beads in a droplet is less than one and/or the number of target entities in a droplet is less than one).

As described herein, a gel bead can comprise a solid support encapsulated therein, for example a single solid support. A single gel bead with a single solid support encapsulated therein can be combined with one or more other components for a reaction. For example, methods described herein relating to reliably encapsulating one gel bead within a droplet can facilitate increased efficiency in combining one gel bead with one or more components for a downstream reaction. Improved reliability in forming droplets comprising a predetermined number of gel beads can improve reliability of pairing the gel beads with desired reagents. In some cases, gel beads can be paired with one or more biological components, including cells. Methods of forming droplets are provided, wherein the droplet contains substantially a single target entity (e.g., a biological cell) and a single gel bead (containing a single solid support). In some embodiments, a majority of the gel beads can each be singly paired within a droplet with a biological cell or entity, or a lysate from a singly encapsulated biological cell or entity. Additionally or in the alternative, the gel beads can be combined with chemical reagents, such as cell lysis reagents, and/or nucleic acid synthesis reagents. The reaction can be performed within the same microfluidic device within which the gel beads and/or the droplets containing the gel beads are formed, or can be in a different microfluidic device.

A method of pairing a gel bead and one or more components for a subsequent reaction can comprise flowing a first fluid stream comprising a plurality of gel beads in a first microfluidic flow channel. The plurality of gel beads can comprise a gel outer layer encapsulating a single solid support. A second fluid stream comprising a plurality of target components can be flowed in a second microfluidic channel. The second fluid stream can be in a direction transverse to that of the first fluid stream such that the first fluid stream intersects the first fluid stream to form a plurality of droplets, where the plurality of droplets comprises a first population of droplets comprising a single gel bead and a single target component. The first population of droplets can be an enriched population. For example, the plurality of droplets can comprise a second population of droplets comprising more than one gel bead, and a third population of droplets comprising no gel beads, the first population of droplets being at a proportion relative to the second and third populations that is greater than that in a Poisson distribution.

### Methods of Labeling Single Cell Entities

The methods provided herein can include delivering a label to a target entity, such as to a single target entity such as a single cell entity. The methods provided herein can further involve accessing the solid support encapsulated within the gel beads, so as to deliver one or more labels on one or more surfaces of the solid support to the target entity. In some embodiments, the gel beads can be softened and/or dissolved to facilitate access of the solid support. For example, the droplet containing the gel bead and the target entity may be subjected to one or more stimuli such that the gelatinous character of the gel outer layer can be reduced for eliminated. The target entity may then be contacted with one or more surfaces of the solid support to facilitate labeling of the target entity with one or more labels on the solid support. In some embodiments, the droplets may be agitated to facilitate mixing of the content of the droplets subsequent to softening and/or dissolving of the gel outer layer such that desired contact can occur between the target entities and the solid supports. For example, the target entities may comprise nucleic acid, such as from cell lysate, including cell lysate from a single cell. The nucleic acid can contact one or more complementary primers on the surface of the solid support labeled with a barcode identifier and hybridize to the primers.

In some embodiments, a gel bead can be an agarose gel bead such that the gel outer layer is agarose. The droplets containing the agarose gel bead may be heated to soften the agarose and/or provide liquid agarose to provide access to the solid support encapsulated by the agarose. For example, the droplets can be heated such that hydrogen bonds of the agarose gel can be disrupted, disrupting the gel structure of the agarose. In some embodiments, the agarose can be heated until the agarose gel assumes a liquid state, for example dissolving or substantially dissolving the agarose.

In some embodiments, the gel structure of the gel bead is not disrupted to provide access to the solid support encapsulated therewithin. For example, one or more components in the droplet can diffuse through pores of the gel outer layer to access one or more chemical moieties on the surfaces of the solid support. For example, the gel structure of a gel bead comprising a polyacrylamide outer layer may not be disrupted to provide access to the solid support encapsulated by the polyacrylamide. Features of the gel beads may not be chemical and/or physically modified to provide access to the solid support, thereby reducing the complexity of the process. The target entity, such as nucleic acid from cell lysate, including nucleic acid from the lysate of a single cell, can diffuse through the pores of the gel outer layer to the solid support. In some embodiments, maintaining the integrity of the gel structure may improve protection of the solid support from impact, thereby reducing or prevent fracture of the solid support. In some embodiments, maintaining the integrity of the gel structure may protect the solid support from friction with other surfaces, including surfaces of other solid supports and/or interior surfaces of the microfluidic device, which can undesirably strip attached molecules from one or more surfaces of the solid support.

A single cell entity includes, variously, a single biological cell or a lysate of a single biological cell as described herein. A single cell entity is often paired with a gel bead containing a single encapsulated solid support or particle. The solid support is often coated with the label.

In a non-limiting example, the label is an oligonucleotide barcode or tag. For example, the solid supports can be coated with oligonucleotides that contain a barcode sequence.

The barcode sequence can be used to tag the nucleic acids from the single cell entities. The barcode may have sufficient specificity such that it can sufficiently tag the nucleic acid. In an example, each solid support within a gel bead is coated with oligonucleotides comprising identical or substantially identical barcode sequences. Barcode sequences amongst different solid supports can vary such that each solid support includes a unique or substantially unique barcode sequence. Single cell entities, such as single cell lysates, can be paired with gel bead-encapsulated solid supports coated with a unique or substantially unique barcode sequence such that each pairing includes a single cell entity and a single, unique or substantially barcode sequence. The oligonucleotides containing the unique or substantially unique barcode sequence can additionally function as primers to e.g., prime a polymerization reaction on a target nucleic acid molecule. The polymerization reaction labels the nucleic acids contained within the single cell entity with the barcode sequence. The nucleic acids may be variously DNA, RNA or both. The oligonucleotides, when serving as primers for a polymerization reaction, hybridize to a complementary sequence on nucleic acids contained within the single cell entity. Various methods of primer extension and polymerization reactions are well known in the art and are consistent with the methods provided herein. Polymerization reactions require various reagents, including, without limitation, buffers, nucleotides or analogues thereof, polymerase enzymes, and the like, and these reagents are introduced, in certain examples, by flowing the reagents in the aqueous stream containing the gel beads. Any reagents necessary or suitable for use in a polymerization reaction are consistent with the methods provided herein.

The droplets can contain reagents suitable for performing a reverse transcription reaction. Non-limiting examples of reagents suitable for performing a reverse transcription reaction, and consistent with the methods provided herein, include buffers, dNTPS, primers, reverse transcriptase enzymes, RNAse inhibitors and the like. In an example, a reverse transcription reaction is performed within a droplet. In this example, the oligonucleotides coated on the solid supports contain a primer sequence that hybridizes to a complementary sequence on mRNA molecules within the single cell entity. Various complementary sequences are found on the oligonucleotide primers that can be used alternately or in combination, including, a poly-T sequence that can hybridize to the poly-A tail of mRNA, a target-specific sequence that is complementary to a target sequence found in the mRNA of a single cell entity, or a random sequence (e.g., a random hexamer). The oligonucleotide primers can further include a barcode sequence and individual droplets containing a single solid support can contain a unique barcode sequence. The primers hybridize to the mRNA contained within the single cell entity and, through the use of a reverse transcriptase enzyme, the mRNA is reverse transcribed such that each resulting complementary DNA (cDNA) has a barcode sequence appended to an end.

After nucleic acids have been labeled with the barcode sequences, the emulsions (e.g., droplets) are broken and the nucleic acids can be pooled. The barcoded nucleic acids are utilized in downstream applications, such as, for example, sequencing methods to identify the source of the nucleic acids. The nucleic acid sequences can be grouped by the barcode sequence to identify nucleic acids that were contained in the same droplet, and hence, were originally contained within the same cell source.

### Additional Applications

The methods described herein are particularly well suited for labeling individual cell entities. Essentially any label or molecule may be delivered to a single target entity utilizing the methods provided herein.

In some embodiments, solid supports are coated with oligonucleotides containing sequences suitable for nucleic acid amplification. For example, the solid supports can be coated with oligonucleotide primers comprising sequence complementary to a target nucleic acid sequence of the single target entity. Nucleic acid amplification reactions, such as polymerase chain reaction (PCR), are performed within the droplets. The solid supports can be coated with one or more oligonucleotide probes that are utilized for gene expression profiling experiments, such as Taqman® probes.

In another non-limiting example, gel beads can contain a transposon for genome fragmentation as a precursor for next-generation sequencing. For example, a transposable element (e.g., a transposon) can be loaded onto a solid support. A transposase enzyme (such as Tn5) can be bound to the transposable element coated on the solid support such that pairing of a gel bead (containing a solid support) with a single cell entity delivers the transposable element and transposase enzyme to the single cell entity. Alternately, the transposable element is loaded onto the solid support and the transposase enzyme is introduced to the droplet after pairing of the gel bead and the single cell entity.

In another non-limiting example, solid supports are coated with affinity molecules. Affinity molecules include, without limitation, antigens, antibodies or aptamers with specific binding affinity for a target molecule. The affinity molecules bind to one or more targets within the single cell entities. Affinity molecules are often detectably labeled (e.g., with a fluorophore). Affinity molecules are sometimes labeled with unique oligonucleotide identifiers. In a non-limiting example, a solid support contains a plurality of affinity molecules, each specific for a different target molecule. Affinity molecules that bind a specific target molecule are collectively labeled with the same oligonucleotide sequence such that affinity molecules with different binding affinities for different targets are labeled with different oligonucleotide sequences. In this way, target molecules within a single target entity are differentially labeled.

In another non-limiting example, solid supports are coated with small molecules, such as drugs or chemical compounds. The methods provided herein often involve pairing gel bead-encapsulated solid supports coated with small molecules with a single target entity. The small molecules are sometimes paired with an oligonucleotide identifier such that the identity of the small molecule delivered to each target entity can be ascertained. Sometimes, the effect of the small molecule on the target cell entity is assayed. For example, the effect of a small molecule on cell viability using an indicator of cell viability (e.g., calcium violet) is determined. In another example, the effect of a small molecule on gene expression is determined (e.g., by simultaneously delivering barcoded primers for reverse transcriptase and measuring mRNA expression).

### Compositions of the Disclosure

Provided herein are compositions for use, alone or in combination with, the methods of the disclosure. The compositions often comprise a solid support or solid particle coated or tagged with a polymer. Various polymers are consistent with the compositions and methods provided herein, and include oligonucleotides, proteins or peptides, nucleic acids, aptamers, affinity molecules, fluorescent markers, and the like. The solid supports or solid particles are encapsulated in a gel or gel-like bead substantially according to methods provided herein.

The solid supports are sometimes spherical or substantially spherical, such as microbeads or microspheres. Solid support compositions consistent with the disclosure include, for example, poly(methyl methacrylate) (PMMA), polystyrene, polyethylene, polypropylene, silica (e.g., glass), or metal. In some embodiments, the solid support can comprise silica. In some embodiments, the solid support can comprise a metal, including one or more of aluminum and steel. Solid supports may be magnetic (e.g., magnetic beads). Solid supports are often flowed through a flow channel of a microfluidic device. The solid supports are often of a size suitable for use with a microfluidic device (e.g., of a size suitable to flow through a microfluidic flow channel). Solid supports often have a size ranging from about 0.5 um to about 200 um in diameter. For example, solid supports sometimes have a diameter of about 0.5 μm, about 0.6 μm, about 0.7 μm, about 0.8 μm, about 0.9 μm, about 1.0 μm, about 1.5 μm, about 2.0 μm, about 2.5 μm, about 3.0 μm, about 3.5 μm, about 4.0 μm, about 4.5 μm, about 5.0 μm, about 5.5 μm, about 6.0 μm, about 6.5 μm, about 7.0 μm, about 7.5 μm, about 8.0 μm, about 8.5 μm, about 9.0 μm, about 9.5 μm, about 10.0 μm, about 20 μm, about 30 μm, about 40 μm, about 50 μm, about 60 μm, about 70 μm, about 80 μm, about 90 μm, about 100 μm, about 120 μm, about 140 μm, about 160 μm, about 180 μm, about 200 μm or greater than about 200 μm. Solid supports sometimes can have a diameter of about 1-200 μm, about 10-70 μm, about 20-60 μm, about 30-50 μm, or about 40 μm. Solid supports often have a diameter of 0.5 μm, 0.6 μm, 0.7 μm, 0.8 μm, 0.9 μm, 1.0 μm, 1.5 μm, 2.0 μm, 2.5 μm, 3.0 μm, 3.5 μm, 4.0 μm, 4.5 μm, 5.0 μm, 5.5 μm, 6.0 μm, 6.5 μm, 7.0 μm, 7.5 μm, 8.0 μm, 8.5 μm, 9.0 μm, 9.5 μm, 10.0 μm, 20 μm, 30 μm, 40 μm, 50 μm, 60 μm, 70 μm, 80 μm, 90 μm, 100 μm, 120 μm, 140 μm, 160 μm, 180 μm, 200 μm or greater than 200 μm. Solid supports can have a diameter of 1-200 μm, 10-70 μm, 20-60 μm, 30-50 μm, or 40μm.

The surface of the solid supports are often functionalized with one or more chemical moieties. Non-limiting examples of functional groups consistent with the disclosure include: alkyl, alkenyl, alkynyl, phenyl, halo, fluoro, chloro, bromo, iodo, hydroxyl, carbonyl, aldehyde, haloformyl, carbonate ester, carboxylate, carboxyl, ester, methoxy, hydroperoxy, peroxy, ether, hemiacetal, hemiketal, acetal, ketal, orthoester, methylenedioxy, orthocarbonate ester, carboxamide, primary amine, secondary amine, tertiary amine, quarternary amine, primary ketimine, secondary ketimine, primary aldimine, secondary aldimine, imide, azide, azo, cyanate, isocyanate, nitrate, nitrile, isonitrile, nitrosooxy, nitro, nitroso, oxime, pyridyl, sulfhydryl, sulfide, disulfide, sulfinyl, sulfonyl, sulfino, sulfo, thiocyanate, isothiocyanate, carbonothioyl, phosphino, phosphono, phosphate, borono, boronate, borino, and borinate. The surfaces of the solid supports are often functionalized to aid in binding or coating of the surface with one or more reagents. The surfaces of the solid supports are often functionalized to allow a chemical reaction to take place on the surface of the solid support.

Alternately or in combination, the surface of the solid supports is optionally coated with one or more reagents. Coating reagents include but are not limited to proteins, oligonucleotides or other nucleic acids, molecular tags, fluorophores, or other markers, alone or in combination. In some examples, solid supports comprise at least one unique molecular tag or barcode identifier. Some solid supports are coated with at least one oligonucleotide that comprises a series of bases having a sequence that functions as a molecular tag or barcode identifier. Some such identifiers are unique in that they occur only once in a population of solid supports. Some identifiers contain sufficient information to allow commonly tagged targets to be confidently mapped to a single source. Alternately, some surfaces comprise heterogeneous populations of identifiers that, in combination, convey sufficient information to allow commonly tagged targets to be confidently mapped to a single source even when individual identifiers do not comprise sufficient information for such mapping. Some solid supports are coated by at least one population of oligonucleotide primer pairs. The oligonucleotide primers often comprise a nucleic acid sequence that is complementary to a nucleic acid sequence on a target molecule.

The solid supports are often rigid (e.g., have a high elastic modulus). The elastic modulus of the solid support can depend on its compositions. For example, the solid supports as envisioned herein have an elastic modulus between about 0.5 to about 200 GPa, including about 0.5 GPa to about 150 GPa, about 0.5 GPa to about 100 GPa, about 0.5 Gpa to about 80 GPa, about 0.5 GPa to about 70 GPa, about 0.5 GPa to about 60 GPa, about 0.5 GPa to about 50 GPa, about 0.5 GPa to about 1 GPa, about 0.5 GPa to about 2 GPa, about 0.5 GPa to about 3 GPa, about 0.5 GPa to about 4 GPa, about 0.5 GPa to about 5 GPa, and about 1 GPa to about 5 GPa, about 2 GPa to about 5 GPa, about 3 GPa to about 5 GPa, about 5 GPa to about 10 GPa, about 5 GPa to about 20 GPa, about 5 GPa to about 40 GPa, or about 5 GPa to about 50 GPa. In some embodiments, the elastic modulus can be about 0.5 GPa, about 1 GPa, about 2 GPa about 3 GPa, about 4 GPa, or about 5 GPa. The elastic modulus can be about 65 GPa, about 67 GPa, about 68 GPa, about 69 GPa, or about 70 GPa. In some embodiments, the elastic modulus can be about 200 GPa.

The compositions often include a gel or gel-like bead. The gel or gel-like bead is often composed of a hydrogel, a polyacrylamide gel, or an agarose gel, or combinations thereof. In some embodiments, gel or gel-like outer layer comprises a superabsorbent polymer. In some embodiments, the gel or gel-like outer layer consists or consists essentially of a superabsorbent polymer. In some embodiments, a superabsorbent polymer is formed by polymerization of acrylic acid blended with sodium hydroxide to form a poly-acrylic acid sodium salt, such as sodium polyacrylate. In some embodiments, a superabsorbent polymer is made using one or more of a polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile.

A gel-precursor fluid is often converted to a gelatinous state to form the gel or gel-like bead, substantially as described herein. The compositions often include a solid support or solid particle encapsulated within the gel bead.

The compositions optionally include a population of gel or gel-like beads. The population of gel or gel-like beads often includes an enriched population of gel or gel-like beads. The enriched population of gel or gel-like beads often includes a majority of gel or gel-like beads with no more than one solid support contained therein. For example, the compositions include an enriched population of gel or gel-like beads, wherein at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or greater than 99% of the gel or gel-like beads encapsulate a single solid support or solid particle.

The gel or gel-like bead is often deformable (e.g., a deformable bead or deformable particle). The deformable beads such as gel beads as envisioned herein can have an elastic modulus of about 0.01 kPa to about 100 kPa. In some embodiments, the elastic modulus of gel beads can be about 0.01 kPa to about 0.1 kPa, or about 0.1 kPa to about 1 kPa. In some embodiments, the compositions provided herein include deformable beads (e.g., gel beads) with an elastic modulus of about 1-10 kPa. For example, the deformable beads have an elastic modulus of about 0.5 kPa, about 1.0 kPa, about 1.5 kPa, about 2.0 kPa, about 2.5 kPa, about 3.0 kPa, about 3.5 kPa, about 4.0 kPa, about 4.5 kPa, about 5.0 kPa, about 5.5 kPa, about 6.0 kPa, about 6.5 kPa, about 7.0 kPa, about 7.5 kPa, about 8.0 kPa, about 8.5 kPa, about 9.0 kPa, about 9.5 kPa, about 10.0 kPa or greater than about 10.0 kPa. Sometimes, deformable beads have an elastic modulus of 1-10 kPa. For example, the deformable beads have an elastic modulus of 0.5 kPa, 1.0 kPa, 1.5 kPa, 2.0 kPa, 2.5 kPa, 3.0 kPa, 3.5 kPa, 4.0 kPa, 4.5 kPa, 5.0 kPa, 5.5 kPa, 6.0 kPa, 6.5 kPa, 7.0 kPa, 7.5 kPa, 8.0 kPa, 8.5 kPa, 9.0 kPa, 9.5 kPa, 10.0 kPa or greater than 10.0 kPa. In some embodiments, the elastic modulus of the gel beads can be about 0.1 kPa to about 60 kPa, about 1 kPa to about 60 kPa, about 10 kPa to about 60 kPa, about 20 kPa to about 60 kPa, about 20 kPa to about 40 kPa. The deformable bead often has an elastic modulus that is less than the elastic modulus of a solid support or solid particle contained therein (e.g., the solid support is more rigid than the deformable bead). The deformable gel bead is often packable such that adjacent compressed gel beads do not adhere to one another. The deformable gel bead is often compressible without loss of shape upon removal of a compressible force.

The compositions often include gel or gel-like beads that are spherical or substantially spherical. The gel or gel-like beads often have a size of about 1-200 μm in diameter, about 1-20 μm in diameter, about 10-15 μm in diameter, about 20-50 μm in diameter, about 35-70 μm in diameter, about 50-100 μm or about 100-200 μm in diameter. For example, the deformable beads have a size of about 1 μm, about 5 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 120 μm, about 140 μm, about 160 μm, about 180 μm, about 200 μm or greater than about 200 μm in diameter. Sometimes, the gel or gel-like beads have a size of 1-200 μm in diameter, 1-20 μm in diameter, 10-15 μm in diameter, 20-50 μm in diameter, 35-70 μm in diameter, 50-100 μm or 100-200 μm in diameter. For example, the deformable beads have a size of 1 μm, 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 120 μm, 140 μm, 160 μm, 180 μm, 200 μm or greater than 200 μm in diameter.

Compositions provided herein sometimes include gel or gel-like beads having a size that is greater than the solid support or solid particle contained therein, such that the solid support or solid particle is entirely or substantially entirely encapsulated by the gel or gel-like bead. For example, the gel or gel-like beads have a size that is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100% or more than 100% greater than the solid supports or solid particles contained therein.

### Biological Samples

The biological samples are variously derived from non-cellular entities comprising polynucleotides (e.g., a virus) or from cell-based organisms (e.g., member of archaea, bacteria, or eukarya domains). The biological sample can be a blood sample. The biological sample can be a cell sample such as a cell culture sample. Cell culture samples include cells in suspension or adherent cells that are lifted from a cell culture dish (e.g., by trypsinization). Cell culture samples can be derived from primary cells or cells from an established cell line, among others.

The biological sample is often derived or obtained from a subject, e.g., plants, fungi, eubacteria, archeabacteria, protists, or animals. The subject is often an organism, either a single-celled or multi-cellular organism. The biological sample is isolated initially from a multi-cellular organism in any suitable form. The animal is sometimes a fish, e.g., a zebrafish. The animal is sometimes a mammal. The mammal is sometimes, without limitation, a dog, cat, horse, cow, mouse, rat, or pig. The mammal is sometimes, without limitation, a primate, e.g., a human, chimpanzee, orangutan, or gorilla. The human is male or female. The sample is sometimes derived from a human embryo or human fetus. The human is an infant, child, teenager, adult, or elderly person. The female is sometimes pregnant, suspected of being pregnant, or planning to become pregnant. The sample is sometimes a single or individual cell from a subject and the biological molecules are derived from the single or individual cell. The sample is sometimes an individual micro-organism, or a population of micro-organisms, or a mixture of micro-organisms and host cellular or cell free nucleic acids.

The biological sample is often obtained from a subject (e.g., human subject) who is healthy. The biological sample is sometimes obtained from a subject (e.g., an expectant mother) at least 4, 5, 6, 7, 8, 9, 10, 11, 12, 13, 14, 15, 16, 17, 18, 19, 20, 21, 22, 23, 24, 25, or 26 weeks of gestation. Sometimes, the subject is affected by a genetic disease, is a carrier for a genetic disease or is at risk for developing or passing down a genetic disease, where a genetic disease is any disease that can be linked to a genetic variation such as mutations, insertions, additions, deletions, translocation, point mutation, trinucleotide repeat disorders and/or single nucleotide polymorphisms (SNPs).

The biological sample is sometimes from a subject who has a specific disease, disorder, or condition, or is suspected of having (or at risk of having) a specific disease, disorder or condition. For example, the biological sample is from a cancer patient, a patient suspected of having cancer, or a patient at risk of having cancer. The cancer is, without limitation, e.g., acute lymphoblastic leukemia (ALL), acute myeloid leukemia (AML), adrenocortical carcinoma, Kaposi Sarcoma, anal cancer, basal cell carcinoma, bile duct cancer, bladder cancer, bone cancer, osteosarcoma, malignant fibrous histiocytoma, brain stem glioma, brain cancer, craniopharyngioma, ependymoblastoma, ependymoma, medulloblastoma, medulloeptithelioma, pineal parenchymal tumor, breast cancer, bronchial tumor, Burkitt lymphoma, Non-Hodgkin lymphoma, carcinoid tumor, cervical cancer, chordoma, chronic lymphocytic leukemia (CLL), chronic myelogenous leukemia (CML), colon cancer, colorectal cancer, cutaneous T-cell lymphoma, ductal carcinoma in situ, endometrial cancer, esophageal cancer, Ewing Sarcoma, eye cancer, intraocular melanoma, retinoblastoma, fibrous histiocytoma, gallbladder cancer, gastric cancer, glioma, hairy cell leukemia, head and neck cancer, heart cancer, hepatocellular (liver) cancer, Hodgkin lymphoma, hypopharyngeal cancer, kidney cancer, laryngeal cancer, lip cancer, oral cavity cancer, lung cancer, non-small cell carcinoma, small cell carcinoma, melanoma, mouth cancer, myelodysplastic syndromes, multiple myeloma, medulloblastoma, nasal cavity cancer, paranasal sinus cancer, neuroblastoma, nasopharyngeal cancer, oral cancer, oropharyngeal cancer, osteosarcoma, ovarian cancer, pancreatic cancer, papillomatosis, paraganglioma, parathyroid cancer, penile cancer, pharyngeal cancer, pituitary tumor, plasma cell neoplasm, prostate cancer, rectal cancer, renal cell cancer, rhabdomyosarcoma, salivary gland cancer, Sezary syndrome, skin cancer, nonmelanoma, small intestine cancer, soft tissue sarcoma, squamous cell carcinoma, testicular cancer, throat cancer, thymoma, thyroid cancer, urethral cancer, uterine cancer, uterine sarcoma, vaginal cancer, vulvar cancer, Waldenstrom Macroglobulinemia, or Wilms Tumor. The sample is often derived from the cancer and/or normal tissue from the cancer patient. The biological sample sometimes is biopsy of a tumor. Alternately, the biological sample is a blood sample that comprises circulating tumor cells (CTCs).

The biological sample is derived from and includes a variety of sources, including, without limitation, aqueous humour, vitreous humour, bile, whole blood, blood serum, blood plasma, breast milk, cerebrospinal fluid, cerumen, enolymph, perilymph, gastric juice, mucus, peritoneal fluid, saliva, sebum, semen, sweat, tears, vaginal secretion, vomit, feces, or urine. The biological sample is sometimes obtained from a hospital, laboratory, clinical or medical laboratory. The sample is often taken from a subject.

Often, the biological sample is an environmental sample comprising medium such as water, soil, air, and the like. The biological sample is sometimes a forensic sample (e.g., hair, blood, semen, saliva, etc.). The biological sample is sometimes an agent used in a bioterrorist attack (e.g., influenza, anthrax, smallpox).

The biological sample is often processed to render it competent for performing any of the methods provided herein. For example, the biological sample is dissociated to generate a dissociated cell population. Biological cells or entities are often encapsulated in droplets prior to further processing, in accordance with the methods provided herein. Droplets often contain, on average, no more than a single biological cell or entity. A single biological cell or entity is sometimes lysed or otherwise disrupted within a droplet. Methods of lysing biological cells within droplets consistent with the methods and compositions described herein are described in the art.

The practice of some embodiments disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

### Numbered Embodiments

1. A method of droplet generation, comprising: transporting a first fluid comprising a plurality of gel beads at a controlled distance relative to one another through a first microfluidic channel, a gel bead of the plurality of gel beads comprising a solid support and a gel outer layer encapsulating the solid support; and generating a plurality of droplets comprising a number of droplets encapsulating a single gel bead at a proportion greater than 20% of the plurality of droplets, the generating comprising intersecting the first fluid with an immiscible carrier fluid. 2. The method of embodiment 1, wherein the plurality of gel beads are closely packed. 3. The method of embodiment 1 or 2, wherein the number of droplets is greater than 30% of the plurality of droplets. 4. The method of embodiment 3, wherein the number of droplets is greater than 40% of the plurality of droplets. 5. The method of embodiment 3, wherein the number of droplets is greater than 50% of the plurality of droplets. 6. The method of any one of embodiments 1 to 5, wherein a gel bead of the plurality of closely packed gel beads is in contact with at least one other gel bead of the plurality of closely packed gel beads. 7. The method of any one of embodiments 6, wherein a gel bead of the plurality of closely packed gel beads is in contact with at least two other gel bead of the plurality of closely packed gel beads. 8. The method of any one of embodiments 6, wherein a gel bead of the plurality of closely packed gel beads is in contact with at least three other gel bead of the plurality of closely packed gel beads. 9. The method of any one of embodiments 1-8, wherein the plurality of gel beads comprise gel having a Young's modulus of 0.01 kPa to about 100 kPa. 10. The method of any one of embodiments 1, wherein the plurality of gel beads is buoyant in the first fluid stream. 11. The method of any one of embodiments 1-10, wherein the plurality of gel beads have a density of 800 kg/m3 to 1000 kg/m3. 12. The method of any one of embodiments 1-11, wherein the gel outer layer comprises acrylamide. 13. The method of any one of embodiments 1-12, wherein the gel outer layer comprises agarose. 14. The method of any one of embodiments 1-13, wherein the solid support is tagged using a molecular tag or barcode identifier such that contents of a tagged microfluidic droplet are identifiably mapped to a common source. 15. The method of any one of embodiments 1-14, wherein the plurality of gel beads occupy greater than 30% of a volume of a segment of the first microfluidic channel. 16. The method of any one of embodiments 1-15, wherein the distance is less than a diameter of a gel bead of the plurality of gel beads. 17. The method of any one of embodiments 1-16, further comprising encapsulating the single solid support with a single cell in a droplet. 18. The method of any one of embodiments 1-17, further comprising encapsulating the single solid support with cell lysis reagents in a droplet for performing cell lysis within the droplet. 19. The method of any one of embodiments 1-18, further comprising combining the single solid support with reagents for nucleic acid synthesis in a droplet. 20. The method of any one of embodiments 1-19, further comprising combining the single solid support with reagents for nucleic acid amplification in a droplet. 21. A method of droplet generation, comprising: transporting a first fluid comprising a plurality of closely packed gel beads through a first microfluidic channel, a gel bead of the plurality of closely packed gel beads comprising a solid support and a gel outer layer encapsulating the solid support; and generating a plurality of droplets comprising a number of droplets containing a single gel bead, the generating comprising intersecting an immiscible carrier fluid and the first fluid by flowing the immiscible carrier fluid and the first fluid through a junction, and the plurality of droplets being generated substantially immediately after the junction, wherein the number of droplets is greater than 20% of the plurality of droplets. 22. The method of embodiment 21, wherein the number of droplets is greater than 50% of the plurality of droplets. 23. The method of embodiment 21 or 22, wherein a gel bead of the plurality of closely packed gel beads is in contact with at least two other gel bead of the plurality of closely packed gel beads. 24. The method of any one of embodiments 21-23, wherein the solid support is tagged using a molecular tag or barcode identifier such that contents of a tagged microfluidic droplet are identifiably mapped to a common source. 25. The method of any one of embodiments 21-24, wherein the gel outer layer comprises acrylamide. 26. The method of any one of embodiments 21-25, wherein the gel outer layer comprises agarose. 27. The method of any one of embodiments 21-26, wherein the plurality of gel beads comprise gel having a Young's modulus of 0.01 kPa to about 100 kPa. 28. The method of any one of embodiments 21-27, wherein the plurality of gel beads is buoyant in the first fluid stream. 29. The method of any one of embodiments 21-28, wherein the plurality of gel beads have a density of 800 kg/m3 to 1000 kg/m3. 30. A method of droplet generation, comprising: transporting a first fluid comprising a plurality of regularly spaced gel beads through a first microfluidic channel, a gel bead of the plurality of closely packed gel beads comprising a solid support and a gel outer layer encapsulating the solid support; and flowing an immiscible carrier fluid in a second microfluidic channel and intersecting the immiscible carrier fluid with the first fluid comprising the plurality of regularly spaced gel beads to controllably generate a plurality of droplets, the plurality of droplets comprising a number of droplets encapsulating a single gel bead, the number of droplets being greater than 20% the plurality of droplets. 31. A method of droplet generation, comprising: transporting a first fluid comprising a plurality of closely packed gel beads through a first microfluidic channel, a gel bead of the plurality of closely packed gel beads comprising a solid support and a gel outer layer encapsulating the solid support; and generating a plurality of droplets comprising a number of droplets encapsulating a single gel bead at a proportion greater than 20% of the plurality of droplets, the generating comprising intersecting the first fluid with an immiscible carrier fluid and the generating being performed without any sorting of the plurality of droplets. 32. A method of droplet generation, comprising: transporting a first fluid comprising a plurality of closely packed gel beads through a first microfluidic channel, a gel bead of the plurality of closely packed gel beads comprising a solid support and a gel outer layer encapsulating the solid support; and flowing an immiscible carrier fluid in a second microfluidic channel and intersecting the immiscible carrier fluid with the first fluid comprising the plurality of closely packed gel beads to generate a plurality of droplets, the plurality of droplets comprising a number of droplets encapsulating a single gel bead, the number of droplets being greater than 20% the plurality of droplets. 33. A method of distributing a population of solid particles to a population of droplets such that a majority of the population of droplets receives a single solid particle per droplet, comprising: flowing a coated population of solid particles in a liquid through a microfluidic channel such that at least half of a segment of a microfluidic channel is occupied by a composition having at least 50% of its volume occupied by coated solid particles; and generating droplets from the composition. 34. The method of embodiment 33, wherein the droplets have a volume that is no more than three times the volume of an average member of the coated population of solid particles. 35. The method of embodiment 33 or 34, wherein the composition has at least 75% of its volume occupied by coated solid particles. 36. The method of any one of embodiments 33-35, wherein a coated solid particle contacts at least three other coated solid particles during flowing. 37. The method of any one of embodiments 33-36, wherein the coating is compressible. 38. The method of any one of embodiments 33-37, wherein the coated particles are neutrally buoyant relative to the liquid. 39. The method of any one of embodiments 33-38, wherein the coated particles are more buoyant that uncoated solid particles. 40. The method of any one of embodiments 33-39, wherein the coated particles are compressible. 41. The method of any one of embodiments 33-40, wherein the coated particles remain suspended in fluid when in contact with one another. 42. The method of any one of embodiments 33-41, wherein the droplets have a volume that is no more than two times the volume of an average member of the coated population of solid particles. 43. The method of any one of embodiments 33-42, wherein the population of droplets is generated without sorting after droplet generation. 44. A method of pairing a gel bead and target components in a droplet, comprising: flowing a first fluid stream comprising a plurality of gel beads, the plurality of gel beads comprising a gel outer layer encapsulating a single solid support; flowing a second fluid stream comprising a plurality of target components for a downstream reaction; and intersecting the first fluid stream and the second fluid stream to form a plurality of droplets, the plurality of droplets comprising a number of droplets encapsulating a single gel bead and a single target component at an efficiency higher than a Poisson distribution. 45. The method of embodiment 44, wherein the plurality of droplets comprises a population of droplets comprising at least one gel bead, and wherein the population of droplets is up to 20% of the plurality of droplets. 46. The method of embodiment 44, wherein the plurality of droplets comprises a population of droplets comprising at least one gel bead, and wherein the population of droplets is between 1% to 20% of the plurality of droplets. 47. The method of embodiment 45 or 46, wherein the number of droplets encapsulating a single gel bead and a single target component is greater than 50% of the population of droplets. 48. The method of embodiment 44, wherein the number of droplets encapsulating a single gel bead and a single target component is at least 1% of the plurality of droplets. 49. The method of embodiment 44, wherein the number of droplets encapsulating a single gel bead and a single target component is at least 2% of the plurality of droplets. 50. The method of embodiment 44, wherein the number of droplets encapsulating a single gel bead and a single target component is at least 5% of the plurality of droplets. 51. The method of embodiment 44, wherein the number of droplets encapsulating a single gel bead and a single target component is at least 10% of the plurality of droplets. 52. The method of any one of embodiments 44-51, wherein the plurality of gel beads are compressible. 53. The method of any one of embodiments 44-52, wherein the plurality of gel beads comprise gel having a Young's modulus of 0.5 GPa to 200 GPa. 54. The method of any one of embodiments 44-53, wherein the plurality of gel beads are densely packed in the first fluid stream such that a bead contacts at least two other beads. 55. The method of any one of embodiments 44-54, wherein the plurality of gel beads is buoyant in the first fluid stream. 56. The method of any one of embodiments 44-55, wherein the plurality of gel beads have a density to provide buoyancy in an aqueous fluid stream. 57. The method of any one of embodiments 44-56, wherein the plurality of gel beads have a density of 500 kg/m3 to 1000 kg/m3. 58. The method of any one of embodiments 44-57, wherein the plurality of target components comprises a plurality of cells. 59. The method of any one of embodiments 44-58, wherein the first fluid stream comprises a first plurality of droplets, at least some of the first plurality of droplets comprising the plurality of gel beads. 60. The method of any one of embodiments 44-59, wherein the second fluid stream comprises a second plurality of droplets, at least some of the second plurality of droplets comprising the plurality of target components. 61. The method of embodiment 60, wherein the second plurality of droplets further comprises reagents for the downstream reaction. 62. The method of any one of embodiments 44-61, wherein the first fluid stream further comprises reagents for the downstream reaction. 63. The method of embodiment 62, wherein the reagents comprise reagents for a nucleic acid synthesis reaction. 64. The method of embodiment 62 or 63, wherein the reagents comprise reagents for a cell lysis reaction. 65. The method of any one of embodiments 44-64, further comprising forming the plurality of gel beads, wherein the forming comprises: flowing a third fluid stream comprising a gel precursor and a plurality of solid supports; flowing a fourth fluid stream in a direction transverse to that of the third fluid stream, the fourth fluid stream comprising an immiscible fluid; intersecting the third fluid stream and the fourth fluid stream to form a plurality of gel precursor beads, at least one of the plurality of gel precursor beads comprising a gel precursor outer layer encapsulating at least one solid support; and stimulating the plurality of gel precursor beads to form a plurality of gel breads. 66. The method of embodiment 65, further comprising sorting the plurality of gel beads to remove gel beads comprising no solid supports and gel beads comprising more than one solid support to provide the plurality of gel beads. 67. The method of embodiment 66, wherein the sorting is based on a respective density of the plurality of gel beads. 68. A method of pairing a gel bead and target components in a droplet, comprising: flowing a first fluid stream comprising a plurality of gel beads, the plurality of gel beads comprising a gel outer layer encapsulating a single solid support; flowing a second fluid stream in a direction transverse to that of the first fluid stream, the second fluid stream comprising a plurality of target components for a downstream reaction; and intersecting the first fluid stream and the second fluid stream to form a plurality of droplets, wherein the plurality of droplets comprises a first population of droplets comprising a single gel bead and a single target component, a second population of droplets comprising more than one gel bead, and a third population of droplets comprising no gel beads, the first population of droplets being at a proportion relative to the second and third populations that is greater than that in a Poisson distribution. 69. An emulsion of droplets comprising gel particles and substrate reagents, wherein at least 5% of the droplets have a single gel particle and a single substrate reagent. 70. The emulsion of embodiment 69, wherein at least 30% of the droplets have a single gel particle and a single substrate reagent. 71. The emulsion of embodiment 69 or 70, wherein at least 50% of the droplets have a single gel particle and a single substrate reagent. 72. The emulsion of any one of embodiments 69-71, wherein at least 75% of the droplets have a single gel particle and a single substrate reagent. 73. The emulsion of any one of embodiments 69-72, wherein the single substrate reagent comprises no more than a single cell lysate. 74. The emulsion of any one of embodiments 69-73, wherein the total number of droplets having a single gel particle and a single substrate reagent is greater than the total number of droplets having less than a single gel particle and a single substrate reagent. 75. The emulsion of any one of embodiments 69-74, wherein the total number of droplets having a single gel particle and a single substrate reagent is greater than the total number of droplets having more than a single gel particle and a single substrate reagent. 76. The emulsion of any one of embodiments 69-75, wherein the total number of droplets having a single gel particle and a single substrate reagent is greater than a sum of the total number of droplets having less than a single gel particle and a single substrate reagent and the total number of droplets having more than a single gel particle and a single substrate reagent. 77. The emulsion of any one of embodiments 69-76, wherein the total number of droplets having a single gel particle is greater than a sum of the total number of droplets having less than a single gel and the total number of droplets having more than a single gel particle. 78. The emulsion of any one of embodiments 69-77, wherein the gel particle comprises a nucleic acid. 79. The emulsion of any one of embodiments 69-78, wherein the gel particle comprises an enzyme. 80. The emulsion of any one of embodiments 69-79, wherein the gel particle comprises a chemical substrate. 81. A method for pairing target beads comprising: (a) providing a plurality of labeling beads, said beads separately comprising a solid support, a label, and a deformable gel coating; and (b) loading the plurality of labeling beads into microfluidic droplets. 82. The method of embodiment 81, wherein individual gel beads are encapsulated into droplets at a frequency not predicted by Poisson statistics. 83. The method of embodiment 81 or 82, comprising discarding microfluidic droplets receiving more than one labeling bead. 84. The method of any one of embodiments 81-83, wherein the solid support comprises PMMA. 85. The method of any one of embodiments 81-84, wherein the solid support comprises polystyrene. 86. The method of any one of embodiments 81-85, wherein the solid support comprises methacrylate. 87. The method of any one of embodiments 81-86, wherein the solid support comprises silica. 88. The method of any one of embodiments 81-87, wherein the solid support comprises a metal. 89. The method of any one of embodiments 81-88, wherein the solid support comprises a binding agent. 90. The method of embodiment 89, wherein the binding agent comprises biotin. 91. The method of embodiment 89 or 90, wherein the binding agent comprises avidin. 92. The method of any one of embodiments 89-91, wherein the binding agent comprises streptavidin. 93. The method of any one of embodiments 89-92, wherein the binding agent comprises a histidine tag. 94. The method of any one of embodiments 89-93, wherein the binding agent comprises nickel ions. 95. The method of any one of embodiments 89-94, wherein the binding agent comprises an antigen. 96. The method of any one of embodiments 89-95, wherein the binding agent comprises an antibody binding region. 97. The method of any one of embodiments 81-96, wherein the plurality of labeling beads are contacted to a cross-linking agent to solidify said deformable gel coating. 98. The method of any one of embodiments 81-97, wherein the plurality of labeling beads are cooled to solidify said deformable gel coating. 99. The method of any one of embodiments 81-98, wherein the plurality of labeling beads are encapsulated within a deformable gel or gel-precursor fluid in an immiscible oil. 100. The method of embodiment 98, wherein the plurality of labeling beads are extracted from the immiscible oil and resuspended in an aqueous phase. 101. The method of any one of embodiments 81-100, wherein the plurality of labeling beads are sorted so as to exclude labeling beads comprising other than one solid support per bead. 102. The method of any one of embodiments 81-102, wherein the plurality of labeling beads are sorted so as to exclude labeling beads comprising other than one label per bead. 103. The method of embodiment 101 or 102, wherein sorting comprises assaying for fluorescence. 104. The method of any one of embodiments 101-103, wherein sorting comprises assaying for light absorption. 105. The method of any one of embodiments 101-104, wherein sorting comprises assaying for magnetic properties. 106. The method of any one of embodiments 101-105, wherein sorting comprises assaying for electrical properties. 107. The method of any one of embodiments 101-106, wherein sorting comprises assaying for density of the solid support. 108. The method of any one of embodiments 101-107, wherein sorting comprises assaying for buoyancy of the solid support. 109. The method of any one of embodiments 101-108, wherein sorting comprises assaying for density of the labeling beads. 110. The method of any one of embodiments 101-109, wherein sorting comprises assaying for buoyancy of the labeling beads. 111. The method of any one of embodiments 101-110, wherein sorting comprises assaying for rigidity of the solid support. 112. The method of any one of embodiments 101-111, wherein a population of gel-encapsulated solid supports is generated having one solid support per gel bead. 113. The method of any one of embodiments 81-112, wherein the solid supports are coated with proteins, oligonucleotides or other nucleic acids. 114. The method of any one of embodiments 81-113, wherein the labeling beads are tagged using a molecular tag or barcode identifier such that contents of a tagged microfluidic droplet are identifiably mapped to a common source. 115. The method of any one of embodiments 81-114, wherein each solid support is tagged using a unique molecular tag or barcode identifier. 116. The method of any one of embodiments 81-115, wherein the gel comprises acrylamide. 117. The method of any one of embodiments 81-116, wherein the gel comprises agarose. 118. The method of any one of embodiments 81-117, wherein the gel comprises a hydrogel. 119. The method of any one of embodiments 81-118, wherein the microfluidic droplets comprise no more than one cell contents unit per droplet. 120. The method of any one of embodiments 81-119, wherein the microfluidic droplets comprise no more than one cell lysate per droplet. 121. A composition comprising a solid support tagged by at least one polymer, wherein the solid support is encased in a semi-solid gel. 122. The composition of embodiment 121, wherein the solid support comprises at least one of PMMA, polystyrene, methacrylate, silica, a metal, or a similar substance. 123. The composition of embodiment 121 or 122, wherein the solid support comprises PMMA. 124. The composition of any one of embodiments 121-123, wherein the solid support comprises polystyrene. 125. The composition of any one of embodiments 121-124, wherein the solid support comprises methacrylate. 126. The composition of any one of embodiments 121-125, wherein the solid support comprises silica. 127. The composition of any one of embodiments 121-126, wherein the solid support comprises a metal. 28. The composition of any one of embodiments 121-127, wherein the solid support comprises a binding agent. 129. The composition of embodiment 128, wherein the binding agent comprises biotin. 130. The composition of embodiment 128 or 129, wherein the binding agent comprises avidin. 131. The composition of any one of embodiments 128-130, wherein the binding agent comprises streptavidin. 132. The composition of any one of embodiments 128-131, wherein the binding agent comprises a histidine tag. 133. The composition of any one of embodiments 128-132, wherein the binding agent comprises nickel ions. 134. The composition of any one of embodiments 128-133, wherein the binding agent comprises an antigen. 135. The composition of any one of embodiments 128-134, wherein the binding agent comprises an antibody binding region. 136. The composition of any one of embodiments 121-135, wherein the solid support has a diameter of from 1-200 um. 137. The composition of any one of embodiments 121-136, wherein the solid support has a diameter of from 5-100 um. 138. The composition of any one of embodiments 121-137, wherein the solid support has a diameter of from 10-70 um. 139. The composition of any one of embodiments 121-138, wherein the solid support has a diameter of from 20-60 um. 140. The composition of any one of embodiments 121-139, wherein the solid support has a diameter of from 30-50 um. 141. The composition of any one of embodiments 121-140, wherein the solid support has a diameter of about 40 um. 142. The composition of any one of embodiments 121-141, wherein the polymer comprises an oligo. 143. The composition of embodiment 142, wherein the oligo uniquely tags the solid support in a population of solid supports. 144. The composition of embodiment 142 or 143, wherein the oligo uniquely tags a microfluidic droplet to which it is contacted. 145. The composition of any one of embodiments 142-144, wherein the oligo tags the solid support in a population of solid supports such that nucleic acids having said oligo are confidently mapped to the solid support. 146. The composition of any one of embodiments 142-145, wherein the oligo tags a microfluidic droplet to which it is contacted such that contents of said microfluidic droplet are confidently mapped to a single source. 147. The composition of any one of embodiments 142-146, wherein the oligo comprises a fluorophore. 148. The composition of any one of embodiments 142-147, wherein the oligo comprises a probe. 149. The composition of any one of embodiments 142-148, wherein the oligo comprises a taqman probe. 150. The composition of any one of embodiments 121-149, wherein the polymer comprises a polypeptide. 151. The composition of embodiment 150, wherein the polypeptide fluoresces when exposed to electromagnetic radiation. 152. The composition of embodiment 150 or 151, wherein the polypeptide fluoresces when exposed to infrared light. 153. The composition of any one of embodiments 150-152, wherein the polypeptide folds to form a GFP barrel structure. 154. The composition of any one of embodiments 150-153, wherein the polypeptide demonstrates at least 90% identity throughout its length to green fluorescent protein. 155. The composition of any one of embodiments 150-154, wherein the polypeptide is a GFP protein. 156. The composition of any one of embodiments 150-155, wherein the polypeptide is a YFP protein. 157. The composition of any one of embodiments 150-157, wherein the polypeptide is a BFP protein. 158. The composition of any one of embodiments 150-158, wherein the polypeptide is an RFP protein. 159. The composition of any one of embodiments 121-158, wherein the composition comprises a fluid, and wherein the solid support encased in the gel results in a gel particle having a buoyancy comparable to that of the fluid. 160. The composition of any one of embodiments 121-159, wherein the composition comprises a fluid, and wherein the solid support encased in the gel results in a gel particle having a buoyancy about that of the fluid. 161. The composition of any one of embodiments 121-160, wherein the solid support encased in the gel results in a gel particle having a size of from 1-200 um. 162. The composition of any one of embodiments 121-161, wherein the solid support encased in the gel results in a gel particle having a size of from 5-100 um. 163. The composition of any one of embodiments 121-162, wherein the solid support encased in the gel results in a gel particle having a size of from 10-70 um. 164. The composition of any one of embodiments 121-163, wherein the solid support encased in the gel results in a gel particle having a size of from 20-60 um. 165. The composition of any one of embodiments 121-164, wherein the solid support encased in the gel results in a gel particle having a size of from 30-50 um. 166. The composition of any one of embodiments 121-165, wherein the solid support encased in the gel results in a gel particle having a size of about 40 um. 167. The composition of any one of embodiments 121-166, wherein the gel is compressible, and wherein the gel particle has an elastic modulus of about 0.01 kPa to about 100 kPa. 168. The composition of any one of embodiments 121-167, wherein adjacent compressed gels do not adhere to one another when suspended in a common fluid in a microfluidic device. 169. A population of gel particles, wherein at least 50% of the gel particles encase a single solid particle per gel particle, said solid particles having at least one polypeptide or oligonucleotide attached thereto. 170. The population of embodiment 169, wherein at least 60% of the gel particles encase a single solid particle per gel particle. 171. The population of embodiment 169 or 170, wherein at least 70% of the gel particles encase a single solid particle per gel particle. 172. The population of any one of embodiments 169-171, wherein at least 80% of the gel particles encase a single solid particle per gel particle. 173. The population of any one of embodiments 169-172, wherein the solid particle comprises PMMA, polystyrene, methacrylate, silica, a metal, or similar substance. 174. The population of any one of embodiments 169-173, wherein the solid support comprises PMMA. 175. The population of any one of embodiments 169-174, wherein the solid support comprises polystyrene. 176. The population of any one of embodiments 169-175, wherein the solid support comprises methacrylate. 177. The population of any one of embodiments 169-176, wherein the solid support comprises silica. 178. The population of any one of embodiments 169-177, wherein the solid support comprises a metal. 179. The population of any one of embodiments 169-178, wherein the solid support comprises a binding agent. 180. The population of embodiment 179, wherein the binding agent comprises biotin. 181. The population of embodiment 179 or 180, wherein the binding agent comprises avidin. 182. The population of any one of embodiments 179-181, wherein the binding agent comprises streptavidin. 183. The population of any one of embodiments 179-182, wherein the binding agent comprises a histidine tag. 184. The population of any one of embodiments 179-183, wherein the binding agent comprises nickel ions. 185. The population of any one of embodiments 179-184, wherein the binding agent comprises an antigen. 186. The population of any one of embodiments 179-185, wherein the binding agent comprises an antibody binding region. 187. The population of any one of embodiments 169-186, wherein the solid particle has a diameter of from 1-200 um. 188. The population of any one of embodiments 169-187, wherein the solid support has a diameter of from 5-100 um. 189. The population of any one of embodiments 169-188, wherein the solid support has a diameter of from 10-70 um. 190. The population of any one of embodiments 169-189, wherein the solid support has a diameter of from 20-60 um. 191. The population of any one of embodiments 169-190, wherein the solid support has a diameter of from 30-50 um. 192. The population of any one of embodiments 169-191, wherein the solid support has a diameter of about 40 um. 193. The population of any one of embodiments 169-192, wherein the solid support has a diameter of 40 um. 194. The population of any one of embodiments 169-193, wherein the solid particle is coated by a population of oligonucleotides. 195. The population of embodiment 194, wherein the population of oligonucleotides uniquely tags a single solid support in the population of solid supports. 196. The population of embodiment 194 or 195, wherein the population of oligonucleotides uniquely tags a microfluidic droplet to which it is contacted. 197. The population of any one of embodiments 194-196, wherein the population of oligonucleotides tags the solid support in a population of solid supports such that nucleic acids having said oligo are confidently mapped to the solid support. 198. The population of any one of embodiments 194-197, wherein the population of oligonucleotides tags a microfluidic droplet to which it is contacted such that contents of said microfluidic droplet are confidently mapped to a single source. 199. The population of any one of embodiments 194-198, wherein the population of oligonucleotides comprises a fluorophore. 200. The population of any one of embodiments 194-199, wherein the population of oligonucleotides comprises a probe. 201. The population of any one of embodiments 194-200, wherein the population of oligonucleotides comprises a taqman probe. 202. The population of any one of embodiments 169-201, wherein the solid particle is coated by a population of polypeptides. 203. The population of embodiment 202, wherein the population of polypeptides fluoresces when exposed to electromagnetic radiation. 204. The population of embodiment 202 or 203, wherein the population of polypeptides fluoresces when exposed to infrared light. 205. The population of any one of embodiments 202-204, wherein the population of polypeptides comprises polypeptides that fold to form a GFP barrel structure. 206. The population of any one of embodiments 202-205, wherein the population of polypeptides demonstrate at least 90% identity throughout its length to green fluorescent protein. 207. The population of any one of embodiments 202-206, wherein the population of polypeptides comprise a GFP protein. 208. The population of any one of embodiments 202-207, wherein the population of polypeptides comprise a YFP protein. 209. The population of any one of embodiments 202-208, wherein the population of polypeptides comprise a BFP protein. 210. The population of any one of embodiments 202-209, wherein the population of polypeptides comprise an RFP protein. 211. The population of any one of embodiments 169-210, wherein the gel particles comprise acrylamide. 212. The population of any one of embodiments 169-211, wherein the gel particles comprise agarose. 213. The population of any one of embodiments 169-212, wherein the gel particles has a density to provide desired buoyancy in a carrier fluid. 214. The population of any one of embodiments 169-213, wherein the gel particles have a diameter of from 1-200 um. 215. The population of any one of embodiments 169-214, wherein the gel particles have a diameter of from 5-100 um. 216. The population of any one of embodiments 169-215, wherein the gel particles have a diameter of from 10-70 um. 217. The population of any one of embodiments 169-216, wherein the gel particles have a diameter of from 20-60 um. 218. The population of any one of embodiments 169-217, wherein the gel particles have a diameter of from 30-50 um. 219. The population of any one of embodiments 169-218, wherein the gel particles have a diameter of about 40 um. 220. The population of any one of embodiments 169-219, wherein the gel particles have a diameter of 40 um. 221. A method of generating a population of gel particles, wherein at least 50% of the gel particles encase a single solid particle per gel particle, said method comprising: (i) providing a plurality of solid particles to a gel precursor liquid; (ii) separating the gel precursor liquid into droplets; (iii) solidifying the gel precursor liquid to form a plurality of gel particles; and (iv) sorting the plurality of gel particles such that gel particles comprising one solid particle per gel particle are separated from the plurality of gel particles. 222. The method of embodiment 221, wherein at least 60% of the gel particles encase a single solid particle per gel particle. 223. The method of embodiment 221 or 222, wherein at least 70% of the gel particles encase a single solid particle per gel particle. 224. The method of any one of embodiments 221-223, wherein at least 80% of the gel particles encase a single solid particle per gel particle. 225. The method of any one of embodiments 221-224, wherein the population of gel particles is compressible such that gel particles do not clump to one another when flowing through a liquid medium. 226. The method of any one of embodiments 221-225, wherein the population of gel particles share a buoyancy comparable to an aqueous liquid compatible with molecular biological manipulations such as reverse transcription or nucleic acid amplification. 227. The method of any one of embodiments 221-226, wherein the solid particle comprises PMMA, polystyrene, methacrylate, silica, a metal, or a similar substance. 228. The method of any one of embodiments 221-227, wherein the solid support comprises PMMA. 229. The method of any one of embodiments 221-228, wherein the solid support comprises polystyrene. 230. The method of any one of embodiments 221-229, wherein the solid support comprises methacrylate. 231. The method of any one of embodiments 221-230, wherein the solid support comprises silica. 232. The method of any one of embodiments 221-231, wherein the solid support comprises a metal. 233. The method of any one of embodiments 221-232, wherein the solid support comprises a binding agent. 234. The population of embodiment 233, wherein the binding agent comprises biotin. 235. The population of embodiment 233 or 234, wherein the binding agent comprises avidin. 236. The population of any one of embodiments 233-235, wherein the binding agent comprises streptavidin. 237. The population of any one of embodiments 233-236, wherein the binding agent comprises a histidine tag. 238. The population of any one of embodiments 233-237, wherein the binding agent comprises nickel ions. 239. The population of any one of embodiments 233-238, wherein the binding agent comprises an antigen. 240. The population of any one of embodiments 233-239, wherein the binding agent comprises an antibody binding region. 241. The method of any one of embodiments 221-240, wherein the gel comprises a hydrogel. 242. The method of any one of embodiments 221-241, wherein the gel comprises a polyacrylamide gel. 243. The method of any one of embodiments 221-242, wherein the gel comprises an agarose gel. 244. The method of any one of embodiments 221-243, wherein the solidifying comprises crosslinking. 245. The method of any one of embodiments 221-244, wherein the solidifying comprises cooling. 246. The method of any one of embodiments 221-245, wherein sorting comprises assaying for fluorescence of the solid support. 247. The method of any one of embodiments 221-246, wherein sorting comprises assaying for light absorption of the solid support. 248. The method of any one of embodiments 221-247, wherein sorting comprises assaying for magnetic properties of the solid support. 249. The method of any one of embodiments 221-249, wherein sorting comprises assaying for electrical properties of the solid support. 250. The method of any one of embodiments 221-249, wherein sorting comprises assaying for buoyancy of the solid support. 251. The method of any one of embodiments 221-250, wherein sorting comprises assaying for density of the solid support. 252. The method of any one of embodiments 221-251, wherein sorting comprises assaying for density of the bead. 253. The method of any one of embodiments 221-252, wherein sorting comprises assaying for buoyancy of the bead. 254. The method of any one of embodiments 221-253, wherein sorting comprises assaying for rigidity of the solid support. 255. A method of delivering a label to a single isolated cell unit comprising the steps of: (i) obtaining a bead comprising the label, a solid particle and an encasing gel exterior; (ii) obtaining an encased isolated cell unit; (iii) suspending the encased isolated cell unit in a fluid; (iv) providing the bead to the fluid; and (v) separating a droplet of the fluid comprising the bead and the encased isolated cell unit; (vi) wherein the bead contacts the encased isolated cell unit to deliver the label to the isolated cell unit. 256. The method of embodiment 255, wherein the label is attached to the solid particle. 257. The method of embodiment 255 or 256, wherein the fluid comprises reagents for nucleic acid amplification. 258. The method of any one of embodiments 255-257, wherein the fluid comprises reagents for reverse transcription. 259. The method of any one of embodiments 255-258, wherein the fluid flows into an opening generating droplets. 260. The method of any one of embodiments 255-259, wherein the cell unit is present at a density of about one per about 1 microliter, about 1 per about 20 microliters, about 1 per about 50 microliters, about 1 per about 100 microliters, about 1 per about 200 microliters, about one per about 100 nanoliters, or about 1 per about 500 nanoliters. 261. The method of any one of embodiments 255-260, wherein the bead is compressible such that upon removal of a compression force a pre-compression shape is restored. 262. The method of any one of embodiments 255-261, wherein the bead is delivered to a reagent flow. 263. The method of any one of embodiments 255-262, wherein the bead possesses a buoyancy comparable to that of the fluid. 264. The method of any one of embodiments 255-263, wherein the bead is present in the fluid at density such that the droplet is at least 50% likely to include no more than 1 bead. 265. The method of any one of embodiments 255-264, wherein the bead is present in the fluid at density such that the droplet is at least 60% likely to include no more than 1 bead. 266. The method of any one of embodiments 255-265, wherein the bead is present in the fluid at density such that the droplet is at least 70% likely to include no more than 1 bead. 267. The method of any one of embodiments 255-266, wherein the bead is present in the fluid at density such that the droplet is at least 80% likely to include no more than 1 bead. 268. The method of any one of embodiments 255-267, wherein the bead comprises a population of oligonucleotides. 269. The method of any one of embodiments 255-268, wherein the population of oligonucleotides uniquely tags a single solid support in the population of solid supports. 270. The method of any one of embodiments 255-269, wherein the population of oligonucleotides uniquely tags a microfluidic droplet to which it is contacted. 271. The method of any one of embodiments 255-270, wherein the population of oligonucleotides tags the solid support in a population of solid supports such that nucleic acids having said oligo are confidently mapped to the solid support. 272. The method of any one of embodiments 255-271, wherein the population of oligonucleotides tags a microfluidic droplet to which it is contacted such that contents of said microfluidic droplet are confidently mapped to a single source. 273. The method of any one of embodiments 255-272, wherein the population of oligonucleotides comprises a fluorophore. 274. The method of any one of embodiments 255-273, wherein the population of oligonucleotides comprises a probe. 275. The method of any one of embodiments 255-274, wherein the population of oligonucleotides a taqman probe. 276. A method of delivering a label to a cell unit, comprising (i) establishing an aqueous reagent stream that exudes droplets from a flow endpoint; (ii) introducing a cell unit into the stream such that nucleic acids of the cell unit do not diffuse throughout the stream prior to being exuded in at least one droplet; (iii) introducing a discrete unit of the label into the stream; and (iv) exuding a droplet comprising the label and at least a portion of the cell unit, (v) wherein the droplet comprises no more than 1 cell unit and no more than 1 label unit. 277. The method of embodiment 276, wherein the aqueous stream comprises reverse transcription reagents. 278. The method of embodiment 276 or 277, wherein the aqueous stream comprises reagents for DNA synthesis. 279. The method of any one of embodiments 276-278, wherein the aqueous stream comprises reagents for polymerase chain reaction-mediated DNA synthesis. 280. The method of any one of embodiments 276-279, wherein the aqueous stream comprises reagents for linear DNA amplification. 281. The method of any one of embodiments 276-280, wherein the aqueous stream comprises cell lysis reagents. 282. The method of any one of embodiments 276-281, wherein the aqueous stream comprises nucleic acid stabilization reagents. 283. The method of any one of embodiments 276-282, wherein the discrete unit of the label comprises an oligonucleotide population. 284. The method of embodiment 283, wherein the oligonucleotide population uniquely tags a single solid support in the population of solid supports. 285. The method of embodiment 283 or 284, wherein the oligonucleotide population uniquely tags a microfluidic droplet to which it is contacted. 286. The method of any one of embodiments 283-285, wherein the oligonucleotide population tags the solid support in a population of solid supports such that nucleic acids having said oligo are confidently mapped to the solid support. 287. The method of any one of embodiments 283-286, wherein the oligonucleotide population tags a microfluidic droplet to which it is contacted such that contents of said microfluidic droplet are confidently mapped to a single source. 288. The method of any one of embodiments 283-287, wherein the population of oligonucleotides comprises a fluorophore. 289. The method of any one of embodiments 283-288, wherein the population of oligonucleotides comprises a probe. 290. The method of any one of embodiments 283-289, wherein the population of oligonucleotides a taqman probe. 291. The method of any one of embodiments 276-290, wherein the discrete unit of the label comprises a fluorescently labeled nucleic acid. 292. The method of any one of embodiments 276-291, wherein the discrete unit of the label comprises a solid particle encased in a gel. 293. The method of embodiment 292, wherein the solid particle comprises at least one of PMMA, polystyrene, methacrylate, silica and a metal. 294. The method of embodiment 292 or 293, wherein the solid support comprises PMMA. 295. The method of any one of embodiments 292-294, wherein the solid support comprises polystyrene. 296. The method of any one of embodiments 292-295, wherein the solid support comprises methacrylate. 297. The method of any one of embodiments 292-296, wherein the solid support comprises silica. 298. The method of any one of embodiments 292-297, wherein the solid support comprises a metal. 299. The method of any one of embodiments 292-298, wherein the solid support comprises a binding agent. 300. The method of embodiment 299, wherein the binding agent comprises biotin. 301. The method of embodiment 299 or 300, wherein the binding agent comprises avidin. 302. The method of any one of embodiments 299-301, wherein the binding agent comprises streptavidin. 303. The method of any one of embodiments 299-302, wherein the binding agent comprises a histidine tag. 304. The method of any one of embodiments 299-303, wherein the binding agent comprises nickel ions. 305. The method of any one of embodiments 299-304, wherein the binding agent comprises an antigen. 306. The method of any one of embodiments 299-305, wherein the binding agent comprises an antibody binding region. 307. The method of any one of embodiments 292-306, wherein the solid particle is bound to a plurality of oligonucleotides. 308. The method of any one of embodiments 292-307, wherein the solid particle is coated with proteins, oligonucleotides or other nucleic acids. 309. A method of generating a population of distinctly labeled cell units comprising: (i) establishing an aqueous reagent flow that exudes a plurality of droplets from a flow endpoint; (ii) sequentially introducing a plurality of cell units into the aqueous flow such that a single cell unit is exuded into at least one droplet prior to nucleic acids of said single cell unit diffusing such that they come into contact with a second cell unit; (iii) sequentially introducing a plurality of gel-coated tagged solid labels into the flow, such that no more than a single gel coated label is exuded into a droplet in at least 50% of the droplets; (iv) wherein the plurality of gel coated labels distinctly label the plurality of cell units. 310. The method of embodiment 309, single gel coated label is exuded into a droplet in at least 60% of the droplets. 311. The method of embodiment 309 or 310, single gel coated label is exuded into a droplet in at least 70% of the droplets. 312. The method of any one of embodiments 309-311, single gel coated label is exuded into a droplet in at least 80% of the droplets. 313. The method of any one of embodiments 309-312, comprising assaying for the number of gel-coated tagged solid labels in a droplet. 314. The method of embodiment 313, wherein assaying comprises assaying for fluorescence of the solid support. 315. The method of embodiment 313 or 314, wherein assaying comprises assaying for light absorption of the solid support. 316. The method of any one of embodiments 313-315, wherein assaying comprises assaying for magnetic properties of the solid support. 317. The method of any one of embodiments 313-316, wherein assaying comprises assaying for electrical properties of the solid support. 318. The method of any one of embodiments 313-317, wherein assaying comprises assaying for density or buoyancy of the solid support. 319. The method of any one of embodiments 313-318, wherein assaying comprises assaying for density or buoyancy of the bead. 320. The method of any one of embodiments 313-319, wherein assaying comprises assaying for rigidity of the solid support. 321. The method of any one of embodiments 313-320, comprising discarding a droplet having more than one gel-coated tagged solid label. 322. The method of any one of embodiments 309-321, wherein the aqueous flow comprises a flow rate greater than the diffusion rate of nucleic acids in the aqueous flow, such that substantially all nucleic acids added to an end of the aqueous flow are included in a droplet generated by budding from that end of the aqueous flow. 323. The method of any one of embodiments 309-322, wherein the aqueous flow comprises a flow rate greater than the diffusion rate of proteins in the aqueous flow, such that substantially all proteins of a cell lysate added to an end of the aqueous flow are included in a droplet generated by budding from that end of the aqueous flow. 324. The method of any one of embodiments 309-323, wherein plurality of gel coated labels comprises oligos. 325. The method of embodiment 324, wherein the oligo uniquely tags a single solid support in the population of solid supports. 326. The method of embodiment 324 or 325, wherein the oligo uniquely tags a microfluidic droplet to which it is contacted. 327. The method of any one of embodiments 324-326, wherein the oligo tags the solid support in a population of solid supports such that nucleic acids having said oligo are confidently mapped to the solid support. 328. The method of any one of embodiments 324-327, wherein the oligo tags a microfluidic droplet to which it is contacted such that contents of said microfluidic droplet are confidently mapped to a single source. 329. The method of any one of embodiments 309-328, wherein the cell units comprise at least one of viral units, discrete virus particles, virus particle populations, microbes, eubacterial cells, archaeal cells, eukaryotic cells, mammalian cells, human cells, and cancer cells. 330. The method of any one of embodiments 309-329, wherein the gel coated label comprises a solid bead. 331. The method of embodiment 330, wherein the solid bead comprises at least one of as PMMA, polystyrene, methacrylate, silica, a metal, or a similar substance. 332. The method of embodiment 330 or 331, wherein the solid bead comprises PMMA. 333. The method of any one of embodiments 330-332, wherein the solid bead comprises polystyrene. 334. The method of any one of embodiments 330-333, wherein the solid bead comprises methacrylate. 335. The method of any one of embodiments 330-334, wherein the solid bead comprises silica. 336. The method of any one of embodiments 330-335, wherein the solid bead comprises a metal. 337. The method of any one of embodiments 330-336, wherein the solid support comprises a binding agent. 338. The method of embodiment 337, wherein the binding agent comprises biotin. 339. The method of embodiment 337 or 338, wherein the binding agent comprises avidin. 340. The method of any one of embodiments 337-339, wherein the binding agent comprises streptavidin. 341. The method of any one of embodiments 337-340, wherein the binding agent comprises a histidine tag. 342. The method of any one of embodiments 337-341, wherein the binding agent comprises nickel ions. 343. The method of any one of embodiments 337-342, wherein the binding agent comprises an antigen. 344. The method of any one of embodiments 337-343, wherein the binding agent comprises an antibody binding region. 345. The method of any one of embodiments 309-344, wherein at least 50% of the cell units comprise a single gel-coated tagged solid label. 346. The method of any one of embodiments 309-345, wherein at least 60% of the cell units comprise a single gel-coated tagged solid label. 347. The method of any one of embodiments 309-346, wherein at least 70% of the cell units comprise a single gel-coated tagged solid label. 348. The method of any one of embodiments 309-347, wherein at least 80% of the cell units comprise a single gel-coated tagged solid label.

Turning to the figures, one sees the following. At FIG. 1, one sees a droplet generation schematic. An aqueous a liquid flow enters the field from the left. The aqueous flow comprises beads at a density such that to total flow volume represented by the beads is substantially smaller than the volume to the flow contributed by the liquid. At center, the flow encounters a carrier fluid, delivered from the perpendicular channels at top and bottom. The flow is partitioned into an emulsion comprising droplets generated from the flow. The frequency of droplets comprising particles roughly reflects the density of particles in the flow prior to droplet partitioning. The resultant droplet population comprises droplets lacking particles and droplets having a single particle. At a lower frequency, not shown, droplets having two particles are generated, at a lower but nonzero frequency.

At FIG. 2, one sees an image of a droplet generation device consistent with the schematic in FIG. 1. At both top and bottom, the fluid flows from left to right, and a carrier fluid is introduced at center to partition the flowing fluid at left into an emulsion, at right. Particle abundance in the emulsion at right roughly approximates particle density in the flow, at left.

At FIG. 3, one sees a population of droplets. Solid supports are visible as dark spots in a minority of the droplets.

At FIG. 4, one sees a droplet sorting schematic. A heterogeneous droplet population such as is generated immediately from a droplet generation device is subjected to sorting. Droplets having a single particle are separated from droplets having zero or more than one particle. Sorting is effected any number of ways, such as microfluidic FACS-based, mass based, buoyancy based, other fluorescence based, or through other approaches.

At FIG. 5 one sees a schematic of a uniform droplet population such as is generated through the disclosure herein. Uniform populations are generated in some cases through sorting, such as is depicted and described in the context of FIG. 4, above. Alternately, in some cases uniform droplet populations or substantially uniform droplet populations are generated directly from droplet generation, in the absence of sorting, through the use of densely packed beads in a fluid flow, such as beads generated so as to have a compressible gel coating that facilitates tight bead packing without bead clumping or clogging of the microchannel.

At FIG. 6 one sees an experimental image of densely packed gel beads flowing through a microfluidic system. At lower left, a large, multi-file population of gel beads flow en masse, frequently contacting one another without clumping or clogging the microfluidic system. At left, the gel beads are flowed into double-file channels, and are seen flowing at a regular, high density in the microchannels. Individual gel beads are seen to be in physical contact with up to 5 or more adjacent gel beads, without disrupting microfluidic flow.

At FIG. 7, one sees the generation of a uniform droplet population such as is generated through the disclosure herein. At left, densely packed gel particles in a liquid flow toward a droplet generation junction. The gel beads are densely packed, with individual gel beads contacting multiple adjacent particles in the flow, without clogging or disrupting flow in the microfluidic channel. Following the droplet junction, center, an emulsion of individual droplets is generated. Droplets each comprise a single gel beads in a uniform emulsion, immediately after droplet generation with no further sorting.

At FIG. 8A, one sees densely packed gel beads flowing through a narrowing microfluidic channel. Single beads contact multiple adjacent beads, and compress and reform their uncompressed shapes in response to pressure from adjacent beads or channel walls.

At FIG. 8B, one sees solid supports, uncoated, passing through a microchannel landscape. At the positions indicated by the dark arrows, center and top, the solid supports are seen to clump in channels created by columns in the microfluidic landscape, despite the channels being substantially wider than the solid bead diameter At lower left, one sees a large clump of solid supports, withdrawn from the liquid flow and static in the microfluidic system. This contrasts to FIG. 8A, where one sees gel particles maintaining flow capabilities despite being in frequent contact with one another and the channel walls, and despite flowing through substantially more narrow channel spaces.

### Examples

The following examples are given for the purpose of illustrating various embodiments and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1

A plurality of solid supports comprising one or more chemical moieties on one or more surfaces can be introduced into a microfluidic flow channel of a microfluidic device by flowing the solid supports in a continuous first fluid stream. The solid supports are not encapsulated in a gel outer layer. The first fluid may be an aqueous solution. For example, the solid supports can be transported in the continuous first fluid stream through a first portion of a first channel of the device such that the solid supports can be deliver to an intersection of the first channel with a second channel. A fluid stream immiscible with the first fluid can be flowed in the second channel. The immiscible fluid can segment the first fluid stream to form droplets containing the first fluid and a solid support as the first fluid stream flows through the intersection from the first portion of the first channel to a second portion of the first channel. The solid supports can be closely packed within the first channel, including a portion of the first channel proximate or adjacent to the intersection. For example, at least one of the closely packed solid supports may be in contact with at least one other of the closely packed solid supports. In this example, the solid supports without the gel outer layer can undesirably fall out of suspension within the first fluid. For example, the solid supports may jam or clog the first channel of the microfluidic device, including when the solid supports are densely packed within the first channel microfluidic device. One or more solid supports may contact one or more surfaces of the first channel and become lodged against the surfaces of the first channel, thereby blocking passage of other solid supports through the first channel.

### Example 2

A plurality of solid supports comprising one or more chemical moieties on one or more surfaces can be introduced into a microfluidic flow channel of a microfluidic device by flowing the solid supports in a continuous first fluid stream. The solid supports are not encapsulated in a gel outer layer. The first fluid may be an aqueous solution. For example, the solid supports can be transported in the continuous first fluid stream through a first portion of a first channel of the device such that the solid supports can be deliver to an intersection of the first channel with a second channel. A fluid stream immiscible with the first fluid can be flowed in the second channel. The immiscible fluid can segment the first fluid stream to form droplets containing the first fluid and a solid support as the first fluid stream flows through the intersection from the first portion of the first channel to a second portion of the first channel. The solid supports can be spaced apart from one another within the first channel, including a portion of the first channel proximate or adjacent to the intersection. For example, a solid support can be at a distance of at least three times a longest length (e.g., a diameter) of a solid particle from the nearest solid support. In this example, the solid supports can flow through the first channel and the intersection to form the droplets without jamming the device. The solid supports can flow through the first channel without becoming lodged against any surfaces of the first channel. However, the efficiency at which the formed droplets contain a solid support can be less than desired. The reduced efficiency of encapsulating a solid support in the droplets can be due to the increased spacing between the solid supports. A reduced rate at which droplets encapsulating a solid support are formed can result in increased waste, and increased time used for generating a predetermined number of droplets which comprise a solid support, thereby resulting in increased cost.

### Example 3

A plurality of solid supports comprising one or more chemical moieties on one or more surfaces can be introduced into a microfluidic flow channel of a microfluidic device by flowing the solid supports in a continuous first fluid stream. These solid supports, in contrast to the solid supports described with reference to Examples 1 and 2 above, are encapsulated in a gel outer layer, for example comprising gel beads having outer gel layers encapsulating solid supports. At least some of the gel beads can comprise a single solid support encapsulated within an outer gel layer. The first fluid may be an aqueous solution. For example, the solid supports encapsulated in the gel outer layer can be transported in the continuous first fluid stream through a first portion of a first channel of the device such that the solid supports can be delivered to an intersection of the first channel with a second channel. A fluid stream immiscible with the first fluid can be flowed in the second channel. The immiscible fluid can segment the first fluid stream to form droplets containing the first fluid and a solid support encapsulated in a gel layer as the first fluid stream flows through the intersection from the first portion of the first channel to a second portion of the first channel. The gel beads in the first channel can be at a controlled distance relative to one another. For example, the gel beads can be positioned close to one another, such as being densely packed, in the first channel. A gel bead can be in direct contact with at least one other gel bead when closely packed in the first channel. The gel beads can be closely packed in the first channel proximate or adjacent to the intersection. In this example, the closely packed gel beads can advantageously be flowed through the first channel without falling out of suspension and/or clogging the first channel. The gel beads comprising the solid supports may advantageously be densely packed within the microfluidic device without jamming or clogging any channels of the device. For example, the gel beads may be densely packed within the first channel such that the gel beads each contact one or more surfaces of the first channel and one or more adjacent gel beads without becoming lodged against the surfaces of the channel. The closely packed gel beads can enable controllably forming droplets which encapsulate a gel bead at an increased rate.

The gel beads may subsequently be used to label a target entity, including for example, nucleic acid from lysate of a single cell. For example, the gel beads may be subsequently combined with the target entity in a droplet. The gel beads may be made of a material which provides ready access of the nucleic acid to the one or more chemical moieties on the surfaces of the solid support such that the nucleic acid can be tagged using the one or more chemical moieties (e.g., barcode identifier used to identify the source of the nucleic acid). The gel bead may advantageously be sufficiently porous such that the nucleic acid may access the solid support through diffusion. For example, the nucleic acid can diffuse through the pores of the gel outer layer to access the solid support, without or substantially without any modification to the chemical and/or physical structure of the gel outer layer. A polyacrylamide gel bead may demonstrate sufficiently porosity to allow diffusion of the nucleic acid therethrough. In another example, the structure of the gel outer layer may be disrupted to facilitate access to the solid support. For example, an agarose gel bead may be heated such that the polymer structure of the agarose gel can be disrupted to allow access of the nucleic acid to the solid support.

After nucleic acids have been labeled with barcode identifiers, the emulsions (e.g., droplets) can be broken and the nucleic acids can be pooled. The barcoded nucleic acids can be utilized in downstream applications, such as, for example, sequencing methods to identify the source of the nucleic acids. For example, the nucleic acid sequences can be grouped by the barcode sequence to identify nucleic acids that were contained in the same droplet, and hence, were originally contained within the same cell source.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of droplet generation, comprising:
transporting a first fluid comprising a plurality of gel beads at a controlled distance relative to one another through a first microfluidic channel, a gel bead of the plurality of gel beads comprising a solid support and a gel outer layer encapsulating the solid support; and
generating a plurality of droplets comprising a number of droplets encapsulating a single gel bead at a proportion greater than 20% of the plurality of droplets, the generating comprising intersecting the first fluid with an immiscible carrier fluid.

2. The method of claim 1, wherein the plurality of gel beads are closely packed.

3. The method of claim 1, wherein the number of droplets is greater than 30% of the plurality of droplets.

4. The method of claim 1, wherein the number of droplets is greater than 40% of the plurality of droplets.

5. The method of claim 1, wherein the number of droplets is greater than 50% of the plurality of droplets.

6. The method of claim 1, wherein a gel bead of the plurality of closely packed gel beads is in contact with at least one other gel bead of the plurality of closely packed gel beads.

7. The method of claim 1, wherein a gel bead of the plurality of closely packed gel beads is in contact with at least two other gel bead of the plurality of closely packed gel beads.

8. The method of claim 1, wherein a gel bead of the plurality of closely packed gel beads is in contact with at least three other gel bead of the plurality of closely packed gel beads.

9. The method of claim 1, wherein the plurality of gel beads comprises gel having a Young's modulus of 0.01 kPa to about 100 kPa.

10. The method of claim 1, wherein the plurality of gel beads is buoyant in the first fluid stream.

11. The method of claim 10, wherein the plurality of gel beads has a density of 800 kg/m³ to 1000 kg/m³.

12. The method of claim 1, wherein the gel outer layer comprises acrylamide.

13. The method of claim 1, wherein the gel outer layer comprises agarose.

14. The method of claim 1, wherein the solid support is tagged using a molecular tag or barcode identifier such that contents of a tagged microfluidic droplet are identifiably mapped to a common source.

15. The method of claim 1, wherein the plurality of gel beads occupy greater than 30% of a volume of a segment of the first microfluidic channel.

16. The method of claim 1, wherein the distance is less than a diameter of a gel bead of the plurality of gel beads.

17. The method of claim 1, further comprising encapsulating the single solid support with a single cell in a droplet.

18. The method of claim 1, further comprising encapsulating the single solid support with cell lysis reagents in a droplet for performing cell lysis within the droplet.

19. The method of claim 1, further comprising combining the single solid support with reagents for nucleic acid synthesis in a droplet.

20. The method of claim 19, further comprising combining the single solid support with reagents for nucleic acid amplification in a droplet.

21. A method of droplet generation, comprising:
transporting a first fluid comprising a plurality of closely packed gel beads through a first microfluidic channel, a gel bead of the plurality of closely packed gel beads comprising a solid support and a gel outer layer encapsulating the solid support; and
generating a plurality of droplets comprising a number of droplets containing a single gel bead, the generating comprising intersecting an immiscible carrier fluid and the first fluid by flowing the immiscible carrier fluid and the first fluid through a junction, and the plurality of droplets being generated substantially immediately after the junction,
wherein the number of droplets is greater than 20% of the plurality of droplets.

22. The method of claim 21, wherein the number of droplets is greater than 50% of the plurality of droplets.

23. The method of claim 21, wherein a gel bead of the plurality of closely packed gel beads is in contact with at least two other gel bead of the plurality of closely packed gel beads.

24. The method of claim 21, wherein the solid support is tagged using a molecular tag or barcode identifier such that contents of a tagged microfluidic droplet are identifiably mapped to a common source.

25. The method of claim 21, wherein the gel outer layer comprises acrylamide.

26. The method of claim 21, wherein the gel outer layer comprises agarose.

27. The method of claim 21, wherein the plurality of gel beads comprises gel having a Young's modulus of 0.01 kPa to about 100 kPa.

28. The method of claim 21, wherein the plurality of gel beads is buoyant in the first fluid stream.

29. The method of claim 28, wherein the plurality of gel beads has a density of 800 kg/m³ to 1000 kg/m³.
